(19) United States
(12) Patent Application Publication
(10) Pub. No.: US 2023/0250393 A1
(43) Pub. Date: Aug. 10, 2023

(54) PRE-TREATING CARDIOMYOCYTES WITH ANTI-ARRHYTHMIC DRUGS TO REDUCE ENGRAFTMENT ARRHYTHMIA

(71) Applicant: University of Washington, Seattle, WA (US)

(72) Inventors: Charles E. MURRY, Seattle, WA (US); William Robb MACLELLAN, Seattle, WA (US); Robert Scott THIES, Seattle, WA (US); Kenta NAKAMURA, Seattle, WA (US)

(73) Assignee: University of Washington, Seattle, WA (US)

(21) Appl. No.: 18/099,419

(22) Filed: Jan. 20, 2023

Related U.S. Application Data
(60) Provisional application No. 63/308,161, filed on Feb. 9, 2022.

(51) Int. Cl. C12N 5/077; A01N 1/02

(52) U.S. Cl. CPC C12N 5/0657; A01N 1/0221; C12N 2533/54

(57) ABSTRACT

Described herein are methods and compositions for reducing or preventing arrhythmias associated with or caused by transplantation of cardiomyocytes to cardiac tissue. In particular embodiments, the pre-treatment of in vitro-differentiated cardiomyocytes with amiodarone before administration to cardiac tissue for engraftment reduces or prevents engraftment arrhythmias, and/or reduces the need for adjunctive anti-arrhythmia drugs after cell administration.

### CROSS REFERENCE TO RELATED APPLICATIONS

The application claims benefit under 35 U.S.C. § 119(e) to U.S. Provisional Application No. 63/308,161 filed Feb. 9, 2022, the contents of which are incorporated herein by reference in its entirety.

### TECHNICAL FIELD

The technology described herein relates to compositions and methods for the improved treatment of cardiac injury or disease while reducing or avoiding arrhythmias associated with engraftment of administered cardiomyocytes.

### BACKGROUND

Cardiomyocyte replacement therapy with human pluripotent stem cell-cardiomyocytes (hPSC-CM) can restore heart function after infarction (Chong et al., 2014; Shiba et al., 2016; Liu et al., 2018). However, hPSC-CM elicit cardiac arrhythmias (ibid., Romagnuolo et al., 2019). These engraftment arrhythmias appear shortly after cell transplantation and persist transiently for 3-4 weeks, during which the recipient is at risk for sudden cardiac death and heart failure.

### SUMMARY

The technology described herein relates to the discovery of methods and compositions for preventing or reducing engraftment arrhythmia in a subject, wherein the engraftment arrhythmia arises from the administration of cardiomyocytes derived from pluripotent stem cells.

Accordingly, provided herein in one aspect is a transplant composition comprising in vitro-differentiated cardiomyocytes and amiodarone.

In one embodiment of this aspect and all other aspects provided herein, the composition further comprises a cryopreservative in an amount sufficient to protect viability of the cells upon freezing.

In another embodiment of this aspect and all other aspects provided herein, the in vitro differentiated cardiomyocytes are human cardiomyocytes.

In another embodiment of this aspect and all other aspects provided herein, the cardiomyocytes are differentiated from induced pluripotent stem (iPS) cells, or embryonic stem cells. In another embodiment of this aspect and all other aspects provided herein, the cardiomyocytes are obtained by direct reprogramming of non-cardiomyocytes or the cell cycle-activation of pre-existing cardiomyocytes.

In another embodiment of this aspect and all other aspects provided herein, the iPS cells are derived from a subject who will receive the transplant composition. In another embodiment of this aspect and all other aspects provided herein, the iPS cells are allogeneic cells.

In another embodiment of this aspect and all other aspects provided herein, the cardiomyocytes are obtained by direct reprogramming of non-cardiomyocytes or the cell cycle-activation of pre-existing cardiomyocytes from the transplant recipient.

In another embodiment of this aspect and all other aspects provided herein, the cardiomyocytes are obtained by direct reprogramming of non-cardiomyocytes or the cell cycle-activation of pre-existing cardiomyocytes that are allogenic to the transplant recipient.

In another embodiment of this aspect and all other aspects provided herein, the amiodarone is present at a concentration of 0.3 to 10 μg/ml of culture medium, inclusive. In this embodiment and all others concerning medium, the medium is preferably a defined, serum-free medium, e.g., as known in the art.

In another embodiment of this aspect and all other aspects provided herein, the concentration of amiodarone is in the range of 1.5 to 4 μg/ml, inclusive.

In another embodiment of this aspect and all other aspects provided herein, the cryopreservative is selected from dimethyl sulfoxide (DMSO), glycerol, sucrose, dextrose, trehalose and polyvinylpyrrolidone.

In another embodiment of this aspect and all other aspects provided herein, the in vitro-differentiated cardiomyocytes have been in contact with the amiodarone for 5 minutes to 24 hours.

In another embodiment of this aspect and all other aspects provided herein, the composition further comprises a scaffold (e.g., scaffold of either synthetic or natural material) or extracellular matrix composition.

In another embodiment of this aspect and all other aspects provided herein, the scaffold or extracellular matrix composition comprises one or more of a synthetic hydrogel, hyaluronic acid, proteoglycan, collagen, fibronectin, vitronectin, and fibrin.

In another embodiment of this aspect and all other aspects provided herein, the scaffold or extracellular matrix composition comprises amiodarone.

Also provided herein, in another aspect is a method of preparing a transplant composition, the method comprising: a) contacting in vitro-differentiated cardiomyocytes with amiodarone; b) contacting the amiodarone-contacted cardiomyocytes of (a) with a cryopreservative in a concentration sufficient to protect viability of the cells upon freezing; and c) freezing the cardiomyocytes resulting from step (b), whereby a transplant composition is prepared.

In one embodiment of this aspect and all other aspects provided herein, the in vitro-differentiated cardiomyocytes are human cardiomyocytes.

In another embodiment of this aspect and all other aspects provided herein, the cardiomyocytes are differentiated from induced pluripotent stem (iPS) cells, embryonic stem cells, by direct reprogramming of non-cardiomyocytes, or by cell cycle induction of cardiomyocytes.

In another embodiment of this aspect and all other aspects provided herein, the iPS cells are derived from a subject who will receive the transplant composition. In another embodiment of this aspect and all other aspects provided herein, the iPS cells are allogeneic iPS cells.

In another embodiment of this aspect and all other aspects provided herein, the cardiomyocytes are contacted with amiodarone at a concentration of 0.3 to 10 μg/ml of culture medium, inclusive.

In another embodiment of this aspect and all other aspects provided herein, the concentration of amiodarone is in the range of 1.5 to 4 μg/ml, inclusive.

In another embodiment of this aspect and all other aspects provided herein, step (a) comprises contacting the in vitro-differentiated cardiomyocytes with amiodarone for 0-24 hours before step (b).

In another embodiment of this aspect and all other aspects provided herein, the cryopreservative is selected from dimethyl sulfoxide (DMSO), glycerol, sucrose, dextrose, trehalose, and polyvinylpyrrolidone.

Another aspect provided herein relates to a method of transplanting cardiomyocytes for engraftment in a subject in need thereof, the method comprising: a) receiving in vitro-differentiated cardiomyocytes derived from an iPS cell derived from the subject (or alternatively are derived from an embryonic stem cell or other cardiomyocyte source described herein), wherein the cardiomyocytes have been contacted with amiodarone; and b) administering the cardiomyocytes to cardiac tissue of the subject.

In one embodiment of this aspect and all other aspects provided herein, the method further comprises administering ivabradine to the subject.

In another embodiment of this aspect and all other aspects provided herein, the dosage of ivabradine is 2.5 to 15 mg, BID. In another embodiment, the dosage of ivabradine is, for example, 7.5 mg, BID.

In another embodiment of this aspect and all other aspects provided herein, ivabradine is administered orally.

In another embodiment of this aspect and all other aspects provided herein, the method further comprises administering amiodarone to the subject.

In another embodiment of this aspect and all other aspects provided herein, the amiodarone is administered orally or intravenously.

In another embodiment of this aspect and all other aspects provided herein, the cardiomyocytes were contacted with a crypopreservative, frozen and thawed prior to step (a).

In another embodiment of this aspect and all other aspects provided herein, the cardiomyocytes have been contacted with amiodarone at a concentration of 0.3 to 10 μg/ml of medium, prior to step (a).

In another embodiment of this aspect and all other aspects provided herein, the cardiomyocytes have been contacted with amiodarone at a concentration of 1.5 to 4 μg/ml, inclusive, prior to step (a).

In another embodiment of this aspect and all other aspects provided herein, the cardiomyocytes have been contacted with amiodarone for 0-24 hours before step (a).

In another embodiment of this aspect and all other aspects provided herein, engraftment arrhythmia burden following administering step (b) is reduced relative to that occurring when a preparation of in vitro-differentiated cardiomyocytes that have not been contacted with amiodarone is administered to a subject.

In another embodiment of this aspect and all other aspects provided herein, reduced engraftment arrhythmia comprises one or more of delayed onset, fewer hours per day, shorter duration, and reduced peak heart rate relative to engraftment arrhythmia caused by administration of in vitro-differentiated cardiomyocytes that were not contacted with amiodarone prior to administration.

In another embodiment of this aspect and all other aspects provided herein, the cardiomyocytes are administered in admixture with a scaffold or extracellular matrix composition.

In another embodiment of this aspect and all other aspects provided herein, the scaffold or extracellular matrix composition comprises one or more of a synthetic hydrogel, hyaluronic acid, collagen, fibronectin, vitronectin and fibrin.

In another embodiment of this aspect and all other aspects provided herein, the scaffold or extracellular matrix composition comprises amiodarone.

Another aspect provided herein relates to a method of reducing cardiac arrhythmia caused by the administration of in vitro-differentiated cardiomyocytes, the method comprising contacting in vitro-differentiated cardiomyocytes with amiodarone, and then administering the cardiomyocytes to cardiac tissue, whereby arrhythmia caused by the administration is reduced relative to arrhythmia caused by administering in vitro-differentiated cardiomyocytes that have not been contacted with amiodarone.

In one embodiment of this aspect and all other aspects provided herein, the method further comprises administering ivabradine to the subject after administering the cardiomyocytes. In another embodiment, the pre-treatment of the cardiomyocytes with amiodarone reduces or obviates the need for adjunctive ivabradine after the cells are transplanted. In some embodiments, pre-treatment with amiodarone can, for example, reduce the dose of ivabradine needed to manage engraftment arrhythmia, and/or reduce the duration of such adjunctive ivabradine administration.

In another embodiment of this aspect and all other aspects provided herein, the method further comprises administering amiodarone to the subject after administering the cardiomyocytes. In another embodiment, the pre-treatment of the cardiomyocytes with amiodarone reduces or obviates the need for adjunctive amiodarone administration after the cells are transplanted. In some embodiments, pre-treatment with amiodarone can, for example, reduce the dose of amiodarone needed post-transplant to manage engraftment arrhythmia, and/or reduce the duration of such adjunctive amiodarone administration.

In another embodiment of this aspect and all other aspects provided herein, the cardiomyocytes are administered in admixture with a scaffold or extracellular matrix composition.

In another embodiment of this aspect and all other aspects provided herein, the scaffold or extracellular matrix composition comprises one or more of a synthetic hydrogel, hyaluronic acid, collagen, fibronectin, vitronectin and fibrin.

In another embodiment of this aspect and all other aspects provided herein, the scaffold or extracellular matrix composition comprises amiodarone.

### BRIEF DESCRIPTION OF THE DRAWINGS

This patent or application file contains at least one drawing executed in color. Copies of this patent or patent application publication with color drawing (s) will be provided by the Office upon request and payment of the necessary fee.

FIG. 1 Flowchart of the study design. Phase 1 consisted of nine total subjects, four used to study the natural history of engraftment arrythmia (EA) and five used to screen seven candidate antiarrhythmic agents. Amiodarone and ivabradine were found to have promising signals of effect and advanced for further study. Phase 2 consisted of 19 total subjects: nine assigned to treatment with amiodarone and ivabradine, eight to no treatment and two to infarct with sham transplant and no anti-arrhythmic drug treatment.

FIG. 2 Variable morphologies of engraftment arrhythmia (EA) in a single pig.

Examples of normal sinus rhythm (NSR) and three morphologies of EA resembling accelerated junctional rhythm (AJR), ventricular tachycardia (VT) and accelerated idioventricular rhythm (AIVR) are observed in this single pig (subject 12). Note the variation in rate, electrical axis, and QRS duration. A continuous rhythm recording below exhibits polymorphic EA with QRS complexes varying in rate, duration, and electrical axis. No sustained arrythmias were noted in surgical sham controls.

FIGS. 3A-3B Acute effects of amiodarone and ivabradine on engraftment arrhythmia. Amiodarone was effective as an intravenous bolus to cardiovert engraftment arrythmia to normal sinus or a lower heart rate (FIG. 3A, red line). Ivabradine administered orally significantly slowed EA but did not cardiovert (FIG. 3B). These data supported a combined amiodarone and ivabradine antiarrhythmic strategy for rhythm and rate control of EA.

FIGS. 4A-4B Antiarrhythmic treatment with amiodarone and ivabradine for engraftment arrhythmia in pig. (FIG. 4A) Kaplan-Meier curve for freedom from primary outcome of cardiac death, unstable EA or heart failure was significantly improved with treatment compared to no treatment (p=0.002). Tic marks on treatment line indicate non-cardiac death due opportunistic infection (days 19 and 26) or a planned euthanasia (day 30). (FIG. 4B) Kaplan-Meier curve for overall survival shows statistically borderline improvement with treatment compared to no treatment (p=0.051). *Death due to Pneumocystis pneumonia. **Death due to porcine cytomegalovirus. Abbreviations: CI, 95% confidence interval.

FIGS. 5A-5F Effect of antiarrhythmic treatment on heart rate and arrhythmia burden. Pooled daily average heart rate (FIG. 5A) and pooled daily average arrhythmia burden (FIG. 5B) with treatment (blue) compared to no treatment (red). The difference in heart rate or arrhythmia burden between treatment and no treatment was not significant (NS) by day 30 post-transplantation. Sham transplant (grey) did not induce tachycardia or arrythmia. Subject-level averaged daily heart rate (FIG. 5C) and arrhythmia burden (FIG. 5D) for antiarrhythmic treatment (blue), no treatment (red) and sham transplant (grey). Unexpected death or euthanasia denoted by black symbol. Peak and mean heart rate (FIG. 5E) and peak and mean arrythmia burden. (FIG. 5F) were significantly reduced with treatment (blue) compared to no treatment (red). **p<0.005.

FIG. 6 Transplanted hESC-CM graft interact with a diffuse Purkinje conduction system in the porcine myocardium. hPSC-cardiomyocyte graft marked by human-specific slow skeletal cardiac troponin I (ssTnI, red) interact with Cx40+(white) PFs. High magnification of boxed regions show example of Purkinje-transitional cell-graft (top) and direct Purkinje-graft (bottom) interactions, scale bar 200 μm (left) or 20 μm (right).

FIG. 7 Study timeline for Phase 2 drug trial of chronic amiodarone and adjunctive ivabradine therapy. Myocardial infarction (MI) was induced by 90-minute balloon occlusion of the mid-left anterior descending artery two weeks prior to human embryonic stem cell-derived cardiomyocyte transplantation (day 0). All subjects received multi-drug immunosuppression. Treatment cohort received rate and rhythm control with combined oral amiodarone and adjunctive oral ivabradine.

FIG. 8 Plasma amiodarone levels in pigs. Amiodarone levels were measured in plasma by a custom liquid chromatography-mass spectrometry assay. Chronic oral amiodarone in six pigs was discontinued after achieving electrical maturation and stabilization of engraftment arrythmia. Serum through concentrations of amiodarone were assayed weekly including 3-4 weeks after discontinuation.

FIG. 9 hESC-CM graft histology and location. Left panel: Histological sections stained with picrosirius red to identify collagen (infarct) and fast green to identify viable myocardium. Adjacent sections labeled with human cTnT (brown) identify transplanted hESC-CM graft within unstained porcine myocardium and scar tissue. Both treatment and no treatment sections were obtained on post-transplantation day 42. Right panel: Transplanted hESC-CM grafts were located similarly between treatment (blue closed square) and no treatment (red open circle) and successfully targeted the infarct and peri-infarct regions of the anterior wall

FIGS. 10A-10B Purkinje fibers are distributed in a mesh-like network throughout the native porcine myocardium and are specifically marked by Connexin 40. Subendocardial and intramyocardial connexin 40 (Cx40)+ Purkinje fibers (PFs, white) in transverse section of left ventricular free wall, scale bars 2 mm (FIG. 10A). Intramyocardial PFs are shown with higher magnification insets. Further magnified view of white boxed regions show Cx40 localizes to gap junctions of Purkinje cells that display lower sarcomere content (F-Actin, red) (i.) and lack T-Tubules (WGA, green) (ii.) in contrast to surrounding cardiomyocytes, scale bar 20 μm. Connexin 40 (Cx40) marks Purkinje fibers (PFs) (FIG. 10B). Cx40 localizes to gap junctions of PFs that display lower sarcomere content (F-Actin, reg) and lack T-Tubules (WGA, green) in contrast to surrounding cardiomyocytes, scale bars 20 μm.

### DETAILED DESCRIPTION

Cardiomyocyte replacement therapy with human pluripotent stem cell-cardiomyocytes (hPSC-CM) can restore heart function after infarction (Chong et al., 2014; Shiba et al., 2016; Liu et al., 2018). However, hPSC-CM elicit cardiac arrhythmias (ibid., Romagnuolo et al., 2019). These engraftment arrhythmias appear shortly after cell transplantation and persist transiently for 3-4 weeks, during which the recipient is at risk for sudden cardiac death and heart failure.

To reduce engraftment arrhythmias, clinically approved anti-arrhythmic drugs were tested in the pig, and amiodarone and ivabradine were identified as potential treatments. However, delayed vacularization of hPSC-CM grafts is evident histologically, and could reduce the effectiveness of these (and other) systemically administered anti-arrhythmic drugs.

The methods described herein can include pretreating hPSC-CM with anti-arrhythmia drugs before the cells are transplanted as a mitigation for reduced drug exposure in the weeks after transplantation. It is specifically contemplated herein that cryopreserved hPSC-CM can be thawed and incubated with therapeutic concentrations of anti-arrhythmic drugs for a time to allow equilibrium-based diffusion into the cells. The drug-loaded cells can optionally be washed free of extraneous anti-arrhythmia drugs before transplantation. Alternatively, the drug-loaded cells can be transplanted with the drug incubation solution serving as an excipient. This process is expected to increase the efficiency of anti-arrhythmia drugs beyond what can be achieved with systemic administration alone. This includes augmenting the effects previously demonstrated with amiodarone and ivabradine as well as eliciting effects with other drugs that were not previously found to be effective for engraftment arrhythmia using systemic dosing. The latter include lidocaine, flecainide, propafenone, sotalol and metoprolol.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in cellular and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the term “cardiomyocyte” refers to a cardiac muscle cell. Cardiomyocytes generally comprise phenotypic and/or structural features associated with cardiac muscle (e.g., electrical phenotypes, sarcomeres, actin, myosin and cardiac troponin T expression, etc.). A cardiomyocyte can be a native cardiomyocyte isolated from an organism or a cardiomyocyte that is differentiated from a stem cell or cardiac precursor (e.g., in-vitro differentiated cardiomyocytes).

As used herein the term “human stem cell” refers to a human cell that can self-renew and differentiate to at least one cell type. The term “human stem cell” encompasses human stem cell lines, human-derived induced pluripotent stem (iPS) cells, human embryonic stem cells, human pluripotent cells, human multipotent stem cells, amniotic stem cells, placental stem cells, or human adult stem cells.

As used herein, “in vitro-differentiated cardiomyocytes” refers to cardiomyocytes that are generated in culture, typically, but not necessarily via step-wise differentiation from a precursor cell such as a human embryonic stem cell, an induced pluripotent stem cell, an early mesoderm cell, a lateral plate mesoderm cell or a cardiac progenitor cell. Thus, while cardiomyocytes in vivo are ultimately derived from a stem cell, i.e., during development of a tissue or organism, a stem cell-derived cardiomyocyte as described herein has been created by in vitro differentiation from a stem cell. As used herein, a cell differentiated in vitro from a stem cell, e.g., an induced pluripotent stem (iPS) cell or embryonic stem cell (“ES cell” or “ESC”), is a “stem-cell derived cardiomyocyte” or “in vitro-differentiated cardiomyocyte” if it has expression of cardiac troponin T (cTnT). Where the electrophysiological disturbances of engraftment arrhythmia are anticipated to occur regardless of the differentiation approach used to generate cardiomyocytes, a cardiac progenitor capable of in vitro differentiation to a cardiomyocyte phenotype expressing cTnT is specifically contemplated. Methods for differentiating stem cells in vitro to cardiomyocytes are known in the art and described elsewhere herein.

The term “isolated cell” as used herein refers to a cell that has been removed from an organism in which it was originally found, or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “substantially pure,” with respect to a particular cell population, refers to a population of cells that is at least about 75%, preferably at least about 85%, more preferably at least about 90%, and most preferably at least about 95% pure, with respect to the cells making up a total cell population. That is, the terms “substantially pure” or “essentially purified,” with regard to a population of cardiomyocytes, refers to a population of cells that contains fewer than about 20%, more preferably fewer than about 15%, 10%, 8%, 7%, most preferably fewer than about 5%, 4%, 3%, 2%, 1%, or less than 1%, of cells that are not cardiomyocytes, respectively.

The term “marker” as used herein is used to describe a characteristic and/or phenotype of a cell. Markers can be used, for example, for selection of cells comprising characteristics of interest and can vary with specific cells. Markers are characteristics, whether morphological, structural, functional or biochemical (enzymatic) characteristics of the cell of a particular cell type, or molecules expressed by the cell type. In one aspect, such markers are proteins. Such proteins can possess an epitope for antibodies or other binding molecules available in the art. However, a marker can consist of any molecule found in or on a cell, including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers can be detected by any method available to one of skill in the art. Markers can also be the absence of a morphological characteristic or absence of proteins, lipids etc. Markers can be a combination of a panel of unique characteristics of the presence and/or absence of polypeptides and other morphological or structural characteristics. In one embodiment, the marker is a cell surface marker.

The term “differentiate”, or “differentiating” is a relative term that indicates a “differentiated cell” is a cell that has progressed further down the developmental pathway than its precursor cell. Thus in some embodiments, a stem cell as the term is defined herein, can differentiate to lineage-restricted precursor cells (e.g., a human cardiac progenitor cell or mid-primitive streak cardiogenic mesoderm progenitor cell), which in turn can differentiate into other types of precursor cells further down the pathway (such as a tissue specific precursor, such as a cardiomyocyte precursor), and then to an end-stage differentiated cell, which plays a characteristic role in a certain tissue type, and may or may not retain the capacity to proliferate further. Methods for in vitro differentiation of stem cells to cardiomyocytes are known in the art and/or described herein below.

The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse and teratoma formation assay. Pluripotency is also evidenced by the expression of embryonic stem (ES) cell markers, although the preferred test for pluripotency is the demonstration of the capacity to differentiate into cells of each of the three germ layers.

As used herein, the terms “induced pluripotent stem cell,” “iPSC,” “hPSC,” and “human pluripotent stem cell” are used interchangeably herein and refer to a pluripotent cell artificially derived from a differentiated somatic cell. iPSCs are capable of self-renewal and differentiation into cell fate-committed stem cells, including cells of the cardiac lineages, as well as various types of mature cells.

The term “derived from,” used in reference to a stem cell means the stem cell was generated by reprogramming of a differentiated cell to a stem cell phenotype. The term “derived from,” used in reference to a differentiated cell means the cell is the result of differentiation, e.g., in vitro differentiation, of a stem cell. As used herein, “iPSC-CMs” or “induced pluripotent stem cell-derived cardiomyocytes” are used interchangeably to refer to cardiomyocytes derived from an induced pluripotent stem cell. In some embodiments, the terms “hPSC-CM” or “human pluripotent stem cell derived cardiomyocytes” are used interchangeabley to refer to cardiomyocytes derived from a human pluripotent stem cell.

As used herein, the terms, “maturation” or “mature phenotype” or “mature cardiomyocytes” when applied to cardiomyocytes refers to the phenotype of a cell that comprises a phenotype similar to adult cardiomyocytes and does not comprise at least one feature of a fetal cardiomyocyte. In some embodiments, markers which indicate increased maturity of an in vitro-differentiated cell include, but are not limited to, electrical maturity, metabolic maturity, genetic marker maturity, and contractile maturity.

As used herein, the terms “transplanting,” “administering” or “engraftment” are used in the context of the placement of cells, e.g. stem cells-derived cardiomyocytes, as described herein into a subject, by a method or route which results in at least partial localization of the introduced cells at a desired site, such as a site of injury or repair, such that a desired effect(s) is produced. The cells e.g., cardiac stem or progenitor cells or cardiomyocytes can be implanted directly to the heart or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. As one of skill in the art will appreciate, long-term engraftment of cardiomyocytes is desired as cardiomyocytes do not proliferate to an extent that the heart can heal from an acute injury comprising cell death. In other embodiments, the cells can be administered via an indirect systemic route of administration, such as an intraperitoneal or intravenous route.

As used herein, the term “contacting” when used in reference to a cell, encompasses introducing an agent, surface, scaffold etc. to the cell in a manner that permits physical contact of the cell with the agent, surface, scaffold etc.

As used herein, the term, “cardiac disease” refers to a disease that affects the cardiac tissue of a subject. Non-limiting examples of cardiac diseases include cardiomyopathy, cardiac arrhythmias, myocardial infarction, heart failure, cardiac hypertrophy, long QT syndrome, arrhythmogenic right ventricular dysplasia (ARVD), catecholaminergic polymorphic ventricular tachycardia (CPVT), Barth syndrome, and Duchenne muscular dystrophy.

“Treatment” of a cardiac disorder, a cardiac disease, or a cardiac injury (e.g., myocardial infarction) as referred to herein refers to a therapeutic intervention that enhances cardiac function and/or enhances cardiomyocyte engraftment and/or enhances cardiomyocyte transplant or graft vascularization in a treated area, thus improving the function of e.g., the heart. That is, cardiac “treatment” is oriented to the function of the heart (e.g., enhanced function within an infarcted area), and/or other site treated with the compositions described herein. A therapeutic approach that improves the function of the heart, for example as assessed by measuring left-ventricular end-systolic dimension (LVESD)) or cardiac output, by at least 10%, and preferably by at least 20%, 30%, 40%, 50%, 75%, 90%, 100% or more, e.g., 2-fold, 5-fold, 10-fold or more, up to and including full function, relative to such function prior to such therapy is considered effective treatment. Effective treatment need not cure or directly impact the underlying cause of the heart disease or disorder to be considered effective treatment. In some embodiments, “treatment” refers to the reduction in the presence or duration of arrhythmias, such as engraftment arrthymias, in a subject and successful treatment of such arrhythmias can be assessed by a partial or complete restoration of a normal sinus rhythm (as detected using an ECG).

As used herein, the term “engraftment arrhythmia” refers to a disturbance in cardiac rate or rhythm caused by or related to the introduction or creation of new cardiac muscle in a subject. A key feature of engraftment arrhythmia is the origination of the stimulus from the site of engraftment, rather than from the SAN or AV node (e.g., an ectopic pacemaker at the site of engraftment). A disturbance in rhythm is any recurring or prolonged deviation from a normal sinus rhythm. A disturbance in heart rate includes a deviation of at least 10% (e.g., at least 10%, 20%, 30%, 40%, 50%, 60%, 70%, 80%, 90% or more) up or down in the subject's normal resting heart rate upon induction or introduction of new cardiomyocytes to a subject's cardiac tissue. In one embodiment, engraftment arrhythmia is caused by or related to the introduction of exogenous cardiomyocytes, including, but not limited to in vitro-differentiated cardiomyocytes, to cardiac tissue, e.g., as in a transplant of cardiomyocytes administered, for example, to promote repair of an infarct or to augment cardiac function, e.g., in a cardiomyopathy. In another embodiment, engraftment arrhythmia comprises a heart rate above 100 beats/minute. In another embodiment, the disturbance in cardiac rate or rhythm is prolonged, e.g., lasting more than 5% of the day or observation period. In another embodiment, an engraftment arrhythmia can be detected via an electrocardiogram (ECG) where a variation in rate, duration, or QRS duration is indicative of an engraftment arrhythmia.

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment of any of the aspects, the subject is human. In another embodiment, of any of the aspects, the subject is an experimental animal or animal substitute as a disease model. In another embodiment, of any of the aspects, the subject is a domesticated animal including companion animals (e.g., dogs, cats, rats, guinea pigs, hamsters etc.). A subject can have previously received a treatment for a disease, or has never received treatment for a disease. A subject can have previously been diagnosed with having a disease, or has never been diagnosed with a disease. A subject can be of any age including, e.g., a fetus, a neonate, a toddler, a child, an adolescent, an adult, a geriatric subject etc.

As used herein, the term “scaffold” refers to a structure, comprising a biocompatible material that provides a surface suitable for adherence and proliferation of cells. A scaffold can further provide mechanical stability and support. A scaffold can be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc.

As used herein, a “substrate” refers to a structure, comprising a biocompatible material that provides a surface suitable for adherence and proliferation of cells. A nanopatterned or micropatterned substrate can further provide mechanical stability and support. A substrate can be in a particular shape or form so as to influence or delimit a three-dimensional shape or form assumed by a population of proliferating cells. Such shapes or forms include, but are not limited to, films (e.g., a form with two-dimensions substantially greater than the third dimension), ribbons, cords, sheets, flat discs, cylinders, spheres, 3-dimensional amorphous shapes, etc. A substrate can be nanopatterned or micropatterned to permit the formation of engineered tissues on the substrate.

As used herein, the term “implantable in a subject” refers to any non-living (e.g., acellular) implantable structure that upon implantation does not generate an appreciable immune response in the host organism. Thus, an implantable structure should not for example, be or contain an irritant, or contain LPS etc.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase,” “increases,” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein, the term “shorter duration,” e.g., when used in reference to duration of engraftment arrhythmia, means that the subject experiences a reduced amount of time in engraftment arrhythmia as the term “reduced” is defined herein. As non-limiting examples, a shorter duration of engraftment arrhythmia can include reduction by a statistically significant amount, by at least 10%, at least 20%, at least 30%, at least 40%, at least 50%, at least 60%, at lest 70%, at least 80%, at least 90% or more.

As used herein, a “reduced peak,” e.g., when used in reference to peak heart rate during engraftment arrhythmia, means that the subject experiences a reduced peak heart rate in engraftment arrhythmia as the term “reduced” is defined herein. As non-limiting examples, a reduced peak heart rate during engraftment arrhythmia can include reduction by a statistically significant amount, by at least 10%, at least 20%, at least 30%, at least 40%, at least 50%, at least 60%, at lest 70%, at least 80%, at least 90% or more.

As used herein, the term “delayed onset,” e.g., when used in reference to engraftment arrhythmia or another symptom, refers to a delay of at least 10% in the time before the arrhythmia or other symptom manifests, e.g., after cardiomyocyte administration to cardiac tissue. Delayed onset can include, for example, a delay of 1 hour, 2 hours, 4 hours, 6 hours, 12 hours, 18 hours, 24 hours or more, e.g., 30 hours, 36 hours, 42 hours, 48 hours or more relative to the onset of symptoms in a subject receiving cardiomyocytes that were not pre-treated as described herein.

As used herein, the term “modulates” refers to an effect including increasing or decreasing a given parameter as those terms are defined herein.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with a disease, or a biological sample that has not been contacted with a composition, polypeptide, or nucleic acid encoding such polypeptide as disclosed herein). In some embodiments, the reference level can refer to a normal sinus rhythm as detected using an ECG. For example, the reference level can comprise the normal duration of the QRS complex (or other portion of the sinus rhythm) in a subject having a normal sinus rhythm.

As used herein, an “appropriate control” refers to an untreated, otherwise identical cell, subject, or population (e.g., a biological sample that was not contacted by an agent or composition described herein, or not contacted in the same manner, e.g., for a different duration, as compared to a non-control cell).

The term “statistically significant” or “significantly” refers to statistical significance and generally means a two standard deviation (2SD) or greater difference.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

Cardiovascular Diseases

In some aspects, provided herein are methods for the treatment and/or prevention of a cardiac injury or a cardiac disease or disorder in a subject in need thereof. The methods described herein can be used to treat, ameliorate, prevent or slow the progression of a number of diseases or their symptoms, such as those resulting in pathological damage to the structure and/or function of the heart.

A cardiovascular disease is a disease that affects the heart and/or circulatory system of a subject. Such cardiac diseases or cardiac-related disease include, but are not limited to, myocardial infarction, cardiac arrhythmia, heart failure, atherosclerotic heart disease, cardiomyopathy, congenital heart defect (e.g., non-compaction cardiomyopathy, septal defects, hypoplastic left heart), hypertrophic cardiomyopathy, dilated cardiomyopathy, cardiac hypertrophy, myocarditis, arrhythmogenic right ventricular dysplasia (ARVD), long QT syndrome, catecholaminergic polymorphic ventricular tachycardia (CPVT), Barth syndrome, valvular stenosis, regurgitation, ischemia, fibrillation, polymorphic ventricular tachycardia, and muscular dystrophies such as Duchenne or related cardiac disease, and cardiomegaly.

The term, “cardiac event” refers to an incident of myocardial injury, myocardial infarction, ventricular fibrillation, stenosis, arrhythmia, or the like.

Symptoms of cardiovascular disease can include but are not limited to syncope, fatigue, shortness of breath, chest pain, and palpitations. A cardiovascular disease is generally diagnosed by a physical examination, blood tests, and/or an electrocardiogram (EKG). An abnormal EKG is an indication that the subject has an abnormal cardiac rhythm or cardiac arrhythmia. Methods of diagnosing arrhythmias are known in the art.

Cardiac electrophysiological and contractile function is a tightly controlled process. When ion channel regulation or contractile function is disrupted in a cardiac cell or tissue, this can result in cardiac arrhythmias that can sometimes be deadly. Cardiac diseases remain a leading cause of death worldwide.

Human stem cell derived cardiomyocytes have emerged as a promising treatment for cardiovascular diseases and cardiac injuries sustained from myocardial infarction. However, the functional maturity of in vitro-differentiated cardiomyocytes in existing models is generally lacking and these cardiomyocytes can cause arrhythmias following engraftment. While not wishing to be bound by theory, the introduction of exogenous cardiomyocytes that have their own impulse generating activity has the potential to disturb the closely regulated electrophysiological function of the heart, leading to arrhythmia—while noted for in vitro-differentiated cardiomyocyte grafts, this effect can also occur, for example, when cardiomyocytes derived from other sources are transplanted to cardiac tissue.

In one aspect, described herein are compositions and methods of treating a cardiovascular disease. In another aspect, described herein is a method of avoiding, treating or ameliorating an engraftment arrhythmia in a subject recipient of a cardiac graft of cardiomyocytes, the method comprises: administering to the subject an in vitro-differentiated human cardiomyocyte pre-treated with an anti-arrhythmic agent (e.g., amiodarone) as described herein, a transplant composition comprising e.g., amiodarone as described herein, or contacting cardiac tissue with pharmacologically manipulated cardiomyocytes delivered via a cardiac delivery device as described herein or any combination thereof.

In some embodiments of any of the aspects, the subject has or is at risk for having a cardiovascular disease or a cardiac event.

In some embodiments of any of the aspects, the subject having a cardiovascular disease is in need of, is receiving or has received a cardiac cell graft. In some embodiments, the subject is at risk for, has or is diagnosed with an engraftment arrhythmia.

As further described herein, an engraftment arrhythmia is a novel and aberrant cardiac rhythm or rate that occurs following administration of a graft of cardiac cells or cardiomyocytes. Engraftment arrhythmias are observed after cardiac graft transplantation and generally persist transiently for days to weeks. Engraftment arrhythmia can cause sudden cardiac death and heart failure in the subject.

Cardiomyocytes for Cardiac Engraftment

The compositions and methods described herein use cardiomyocytes that have been pre-treated with one or more anti-arrhythmic agents (e.g., amiodarone) that prevents or reduces electrical disturbances when the cardiomyocytes are engrafted into a subject for the treatment of heart disease or disorder (e.g., myocardial infarction or heart failure).

Cardiac engraftment administers cardiomyocytes to a site of cardiac injury in the heart. A skilled physician can determine the site of injury by methods known in the art. A primary goal of cardiac engraftment is to provide electrical and mechanical stability to the injured myocardium that cannot be achieved by pharmaceutical treatments alone.

The cardiomyocytes described herein can be isolated from a human subject or differentiated from stem cells or a cardiac precursor. The following describes various sources and stem cells that can be used to prepare cardiomyocytes for engraftment into a subject.

Stem cells are cells that retain the ability to renew themselves through mitotic cell division and can differentiate into more specialized cell types. Three broad types of mammalian stem cells include: embryonic stem (ES) cells that are found in blastocysts, induced pluripotent stem cells (iPSCs) that are reprogrammed from somatic cells, and adult stem cells that are found in adult tissues. Other sources of pluripotent stem cells can include amnion-derived or placental-derived stem cells. Pluripotent stem cells can differentiate into cells derived from any of the three germ layers.

Cardiomyocytes useful in the compositions and methods described herein can be differentiated from embryonic stem cells and induced pluripotent stem cells, among others. In one embodiment, the compositions and methods provided herein use human cardiomyocytes differentiated from embryonic stem cells. Alternatively, in some embodiments, the compositions and methods provided herein do not encompass generation or use of human cardiogenic cells made from cells taken from a viable human embryo.

Embryonic stem cells: Embryonic stem cells and methods for their retrieval are well known in the art and are described, for example, in Trounson A O Reprod Fertil Dev (2001) 13: 523, Roach M L Methods Mol Biol (2002) 185: 1, and Smith A G Annu Rev Cell Dev Biol (2001) 17:435. The term “embryonic stem cell” is used to refer to the pluripotent stem cells of the inner cell mass of the embryonic blastocyst (see e.g., U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970).

Cells derived from embryonic sources can include embryonic stem cells or stem cell lines obtained from a stem cell bank or other recognized depository institution. Other means of producing stem cell lines include methods comprising the use of a blastomere cell from an early stage embryo prior to formation of the blastocyst (at around the 8-cell stage). Such techniques correspond to the pre-implantation genetic diagnosis technique routinely practiced in assisted reproduction clinics. In this approach, a single blastomere cell is co-cultured with established ES-cell lines and then separated from them to form fully competent ES cell lines.

Undifferentiated embryonic stem (ES) cells are easily recognized by those skilled in the art, and typically appear in the two dimensions of a microscopic view in colonies of cells with high nuclear/cytoplasmic ratios and prominent nucleoli. In some embodiments, the human cardiomyocytes described herein are not derived from embryonic stem cells or any other cells of embryonic origin.

Induced Pluripotent Stem Cells (iPSCs):

In some embodiments, the compositions and methods described herein utilize cardiomyocytes that are differentiated in vitro from induced pluripotent stem cells. An advantage of using iPSCs to generate cardiomyocytes for the compositions described herein is that, if so desired, the cells can be derived from the same subject to which the desired human cardiomyocytes are to be administered. That is, a somatic cell can be obtained from a subject, reprogrammed to an induced pluripotent stem cell, and then re-differentiated into a human cardiomyocyte to be administered to the subject (e.g., autologous cells). Since the cardiomyocytes (or their differentiated progeny) are essentially derived from an autologous source, the risk of engraftment rejection or allergic responses is reduced compared to the use of cells from another subject or group of subjects. While this is an advantage of iPS cells, in alternative embodiments, the cardiomyocytes useful for the methods and compositions described herein are derived from non-autologous sources (e.g., allogenic). In addition, the use of iPSCs negates the need for cells obtained from an embryonic source. Thus, in one embodiment, the stem cells used to generate cardiomyocytes for use in the methods and compositions described herein are not embryonic stem cells.

Although differentiation is generally irreversible under physiological contexts, several methods have been developed in recent years to reprogram somatic cells to induced pluripotent stem cells. Exemplary methods are known to those of skill in the art and are described briefly herein below.

Reprogramming is a process that alters or reverses the differentiation state of a differentiated cell (e.g., a somatic cell). Stated another way, reprogramming is a process of driving the differentiation of a cell backwards to a more undifferentiated or more primitive type of cell. It should be noted that placing many primary cells in culture can lead to some loss of fully differentiated characteristics. However, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character when differentiated cells are placed in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. Thus, cells can be terminally differentiated somatic cells, as well as from adult stem cells, or somatic stem cells.

In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. In certain embodiments described herein, reprogramming of a differentiated cell (e.g., a somatic cell) causes the differentiated cell to assume an undifferentiated state with the capacity for self-renewal and differentiation to cells of all three germ layer lineages. These are induced pluripotent stem cells (iPSCs or iPS cells).

Methods of reprogramming somatic cells into iPS cells are known in the art. See for example, U.S. Pat. Nos. 8,129,187 B2; 8,058,065 B2; US Patent Application 2012/0021519 A1; Singh et al. Front. Cell Dev Biol. (2015); and Park et al. Nature (2008); which are incorporated by reference in their entireties. Specifically, iPSCs are generated from somatic cells by introducing a combination of reprogramming transcription factors. The reprogramming factors can be e.g., nucleic acids, vectors, small molecules, viruses, polypeptides, or any combination thereof. Non-limiting examples of reprogramming factors include Oct4 (Octamer binding transcription factor-4), Sox2 (Sex determining region Y)-box 2, Klf4 (Kruppel Like Factor-4), and c-Myc. Additional factors (e.g., LIN28+Nanog, Esrrb, Pax5 shRNA, C/EBPa, p53 siRNA, UTF1, DNMT shRNA, Wnt3a, SV40 LT(T), hTERT) or chemicals (e.g., BIX-01294, BayK8644, RG108, AZA, dexamethasone, VPA, TSA, SAHA, PD025901+CHIR99021(2i), A-83-01) have been found to replace one or the other reprogramming factors from basal reprogramming factors or to enhance the efficiency of reprogramming.

The specific approach or method used to generate pluripotent stem cells from somatic cells (e.g., any cell of the body with the exclusion of a germ line cell; fibroblasts, etc.) is not critical to the claimed embodiment(s). Thus, any method that re-programs a somatic cell to the pluripotent phenotype would be appropriate for use in the methods described herein.

Reprogrammed somatic cells as disclosed herein can express any of a number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-III-tubulin; α-smooth muscle actin (α-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fth117; Sal 14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tell); DPPA3/Stella; DPPA4; other general markers for pluripotency, etc. Other markers can include Dnmt3L; Sox15; Stat3; Grb2; β-catenin, and Bmil. Such cells can also be characterized by the down-regulation of markers characteristic of the somatic cell from which the induced pluripotent stem cell is derived.

The efficiency of reprogramming (i.e., the number of reprogrammed cells) derived from a population of starting cells can be enhanced by the addition of various small molecules as shown by Shi, Y., et al. (2008) Cell-Stem Cell 2:525-528, Huangfu, D., et al. (2008) Nature Biotechnology 26(7):795-797, and Marson, A., et al. (2008) Cell-Stem Cell 3:132-135. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

To confirm the induction of pluripotent stem cells for use with the methods described herein, isolated clones can be tested for the expression of one or more stem cell markers. Such expression in a cell derived from a somatic cell identifies the cells as induced pluripotent stem cells. Stem cell markers can include but are not limited to SSEA3, SSEA4, CD9, Nanog, Oct4, Fbx15, Ecat1, Esg1, Eras, Gdf3, Fgf4, Cripto, Dax1, Zpf296, Slc2a3, Rex1, Utf1, and Nat1, among others. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

Adult Stem Cells: Adult stem cells are stem cells derived from tissues of a post-natal or post-neonatal organism or from an adult organism. An adult stem cell is structurally distinct from an embryonic stem cell not only in markers it does or does not express relative to an embryonic stem cell, but also by the presence of epigenetic differences, e.g. differences in DNA methylation patterns. It is contemplated that cardiomyocytes differentiated from adult stem cells can also be used for cardiac grafts as described herein. Methods of isolating adult stem cells are known in the art. See for example, U.S. Pat. No. 9,206,393 B2; and US Application No. 2010/0166714 A1; which are incorporated herein by reference in their entireties.

Pre-Treatment of Cardiomyocytes with Anti-Arrhythmia Agents

Provided herein are methods and compositions for preventing or reducing arrhythmias associated with engrafted cardiomyocytes in a subject. Such methods and compositions relate to the pre-treatment of cardiomyocytes or co-adminstration of cardiomyocytes with anti-arrythmic agents.

In one embodiment, the cardiomyocytes are pre-treated with amiodarone or a combination of amiodarone and ivabradine. In other embodiments, the cardiomyocytes are pre-treated with one or more anti-arrhythmic agents selected from the group consisting of: amiodarone, ivabradine, lidocaine, flecainide, propafenone, sotalol and metoprolol.

In one embodiment, the cardiomyocytes are pre-treated with amiodarone with a dose within the range of 0.3 μg/mL to 12 μg/mL. In some embodiments, the dose of amiodarone used to pre-treat cardiomyocytes comprises 0.4 μg/mL to 12 μg/mL, 0.5 μg/mL to 12 μg/mL, 0.6 μg/mL to 12 μg/mL, 0.7 μg/mL to 12 μg/mL, 0.8 μg/mL to 12 μg/mL, 0.9 μg/mL to 12 μg/mL, 1.0 μg/mL to 12 μg/mL, 1.5 μg/mL to 12 μg/mL, 2.0 μg/mL to 12 μg/mL, 2.5 μg/mL to 12 μg/mL, 3.0 μg/mL to 12 μg/mL, 3.5 μg/mL to 12 μg/mL, 4 μg/mL to 12 μg/mL, 4.5 μg/mL to 12 μg/mL, 5.0 μg/mL to 12 μg/mL, 5.5 μg/mL to 12 μg/mL, 6 μg/mL to 12 μg/mL, 6.5 μg/mL to 12 μg/mL, 7.0 μg/mL to 12 μg/mL, 7.5 μg/mL to 12 μg/mL, 8.0 μg/mL to 12 μg/mL, 8.5 μg/mL to 12 μg/mL, 9 μg/mL to 12 μg/mL, 9.5 μg/mL to 12 μg/mL, 10 μg/mL to 12 μg/mL, 10.5 μg/mL to 12 μg/mL, 11 μg/mL to 12 μg/mL, 11.5 μg/mL to 12 μg/mL, 0.3 μg/mL to 11.5 μg/mL, 0.3 μg/mL to 11 μg/mL, 0.3 μg/mL to 10.5 μg/mL, 0.3 μg/mL to 10 μg/mL, 0.3 μg/mL to 9.5 μg/mL, 0.3 μg/mL to 9 μg/mL, 0.3 μg/mL to 8.5 μg/mL, 0.3 μg/mL to 8 μg/mL, 0.3 μg/mL to 7.5 μg/mL, 0.3 μg/mL to 7 μg/mL, 0.3 μg/mL to 6.5 μg/mL, 0.3 μg/mL to 6 μg/mL, 0.3 μg/mL to 5.5 μg/mL, 0.3 μg/mL to 5 μg/mL, 0.3 μg/mL to 4.5 μg/mL, 0.3 μg/mL to 4 μg/mL, 0.3 μg/mL to 3.5 μg/mL, 0.3 μg/mL to 3 μg/mL, 0.3 μg/mL to 2.5 μg/mL, 0.3 μg/mL to 2 μg/mL, 0.3 μg/mL to 1.5 μg/mL, 0.3 μg/mL to 1 μg/mL, 0.3 μg/mL to 0.8 μg/mL, 0.3 μg/mL to 0.5 μg/mL, or 0.3 μg/mL to 0.4 μg/mL. In other embodiments, the does of amiodarone used to pre-treat cardiomyocytes comprises 1 μg/mL to 10 μg/mL, 1 μg/mL to 8 μg/mL, 1 μg/mL to 5 μg/mL, 1 μg/mL to 4 μg/mL, 1 μg/mL to 3 μg/mL, 1 μg/mL to 2 μg/mL, 1.5 μg/mL to 6 μg/mL, 1.5 μg/mL to 5 μg/mL, 1.5 μg/mL to 4 μg/mL, 1.5 μg/mL to 3 μg/mL, 1.5 μg/mL to 2 μg/mL, 2 μg/mL to 10 μg/mL, 2 μg/mL to 8 μg/mL, 2 μg/mL to 5 μg/mL, 2 μg/mL to 4 μg/mL, 2 μg/mL to 3 μg/mL, 3 μg/mL to 10 μg/mL, 3 μg/mL to 8 μg/mL, 3 μg/mL to 5 μg/mL, 3 μg/mL to 4 μg/mL, or any range therebetween. In certain embodiments, the cardiomyocytes are pre-treated with one or more anti-arrhythmic agents (e.g., amiodarone) and then frozen/cryogenically preserved in at least one cryoprotectant. In such embodiments, the cells are thawed prior to administration to a subject for the treatment of a given cardiac injury, disease or disorder. Thus, provided herein in one embodiment is a composition comprising a therapeutically effective amount of cardiomyocytes (e.g., iPSC-CMs or hPSC-CMs), a therapeutically effective amount of an anti-arrhythmic agent (e.g., amiodarone) and a croprotectant/cryopreservative. Essentially any cryopreservative is contemplated for use with the methods and compositions described herein including, but not limited to, dimethyl sulfoxide (DMSO) a cryopreservative included, for example, in the commercially available products Cryostor™ (StemCell Technologies, Vancouver, BC) and CryoStor Dlite™ (BioLife Solutions, Inc. Bothell, Wash.). Other cryopreservative agents include, but are not imited to glycerol, sucrose, dextrose, trehalose and polyvinylpyrrolidone. High salt cryopreservatives are also contemplated for use herein.

The cardiomyocytes can be pre-treated with a given anti-arrhythmic agent at least 36 h, at least 30 h, at least 24 h, at least 20 h, at least 18 h, at least 15 h, at least 12 h, at least 10 h, at least 8 h, at least 6 h, at least 4 h, at least 2 h, at least 1 h, at least 30 minutes, at least 15 minutes, at least 10 minutes, or at least 5 minutes prior to freezing. In another embodiment, the cardiomyocytes can be pre-treated with a given anti-arrhythmic agent for no more than 36 h, no more than 30 h, no more than 24 h, no more than 20 h, no more than 18 h, no more than 15 h, no more than 12 h, no more than 10 h, no more than 8 h, no more than 6 h, no more than 4 h, no more than 2 h, no more than 1 h, no more than 30 minutes, no more than 15 minutes, no more than 10 minutes, or no more than 5 minutes prior to freezing. In another embodiment, the cells can be admixed with the cryopreservative and the anti-arrhythmic drug and then immediately frozen.

In alternative embodiments, the cardiomyocytes can be frozen, thawed and then loaded with a given anti-arrhythmic agent prior to administration to a subject.

In Vitro-Differentiation

Various methods and compositions described herein use in vitro-differentiated cardiomyocytes. Methods for the differentiation of either cell type from ESCs or iPSCs are known in the art. See, e.g., LaFlamme et al., Nature Biotech 25:1015-1024 (2007), which describes the differentiation of cardiomyocytes which is incorporated herein by reference in its entirety.

In certain embodiments, the step-wise differentiation of ESCs or iPSCs to cardiomyocytes proceeds in the following order: ESC or iPSC>cardiogenic mesoderm>cardiac progenitor cells>cardiomyocytes (see e.g., Lian et al. Nat Prot (2013); US Applicant No. 2017/0058263 A1; 2008/0089874 A1; 2006/0040389 A1; U.S. Pat. Nos. 10,155,927 B2; 9,994,812 B2; and 9,663,764 B2, the contents of each of which are incorporated herein by reference their entireties). A number of protocols for differentiating ESCs and iPSCs to cardiomyocytes are known in the art. For example, agents can be added or removed from cell culture media to direct differentiation to cardiomyocytes in a step-wise fashion. Non-limiting examples of factors and agents that can promote cardiomyocyte differentiation include small molecules (e.g., Wnt inhibitors, GSK3 inhibitors), polypeptides (e.g., growth factors), nucleic acids, vectors, and patterned substrates (e.g., nanopatterns). The addition of growth factors necessary in cardiovascular development, including but not limited to fibroblast growth factor 2 (FGF2), transforming growth factor β (TGFβ) superfamily growth factors—Activin A and BMP4, vascular endothelial growth factor (VEGF), and the Wnt inhibitor DKK-1, can also be beneficial in directing differentiation along the cardiac lineage. Additional examples of factors and conditions that help promote cardiomyocyte differentiation include but are not limited to B27 supplement lacking insulin, cell-conditioned media, external electrical pacing, and nanopatterned substrates, among others.

As will be appreciated by those of skill in the art, in vitro-differentiation of cardiomyocytes produces an end-result of a cell having the phenotypic and morphological features of the desired cell type but the differentiation steps of in vitro-differentiation need not be the same as the differentiation that occurs naturally in the embryo. That is, during differentiation to a cardiomyocyte, it is specifically contemplated herein that the step-wise differentiation approach utilized to produce such cells need not proceed through every progenitor cell type that has been identified during embryogenesis and can essentially “skip” over certain stages of development that occur during embryogenesis; see, e.g., WO2018096343 in regard to transcription factor-mediated reprogramming of hPSCs. It is also contemplated that cardiomyocytes derived from other cells, e.g., via transdifferentiation can also benefit from the modulation of the ion channel set descrived herein when used for transplant.

Monitoring Differentiation of Cardiomyocytes and Functional Characterization

As will be appreciated by one of skill in the art, an in vitro-differentiated cardiomyocyte as described herein will lack markers of hematopoietic or hemogenic cells, vascular endothelial cells, embryonic stem cells or induced pluripotent stem cells. In one embodiment of the methods described herein, one or more cell surface markers are used to determine the degree of differentiation along the spectrum of embryonic stem cells or iPSCs to e.g., fully differentiated cardiomyocytes.

In some embodiments, antibodies or similar agents specific for a given marker, or set of markers, can be used to separate and isolate the desired cells using fluorescent activated cell sorting (FACS), panning methods, magnetic particle selection, particle sorter selection and other methods known to persons skilled in the art, including density separation (Xu et al. (2002) Circ. Res. 91:501; U.S.S.N. 20030022367) and separation based on other physical properties (Doevendans et al. (2000) J. Mol. Cell. Cardiol. 32:839-851). Negative selection can be performed, including selecting and removing cells with undesired markers or characteristics, for example fibroblast markers, epithelial cell markers etc.

Undifferentiated ES cells express genes that can be used as markers to detect the presence of undifferentiated cells. Exemplary ES cell markers include stage-specific embryonic antigen (SSEA)-3, SSEA-4, TRA-I-60, TRA-1-81, alkaline phosphatase or those described in e.g., U.S. S.N. 2003/0224411; or Bhattacharya (2004) Blood 103(8):2956-64, each herein incorporated by reference in their entirety. Exemplary markers expressed on cardiac progenitor cells include, but are not limited to, TMEM88, GATA4, ISL1, MYL4, and NKX2-5.

Exemplary markers expressed on cardiomyocytes include, but are not limited to, NKX2-5, MYH6, MYL7, TBXS, ATP2a2, RYR2, and cTnT.

In some embodiments, the desired cells (e.g., in vitro-differentiated cardiomyocytes) are an enriched population of cells; that is, the percentage of in vitro-differentiated cardiomyocytes (e.g., percent of cells) in a population of cells is at least 10% of the total number of cells in the population. For example, an enriched population comprises at least 15% definitive cardiomyocytes, at least 20%, at least 30%, at least 40%, at least 50%, at least 60%, at least 70%, at least 80%, at least 90%, at least 95%, at least 99% or even 100% of the population comprises human in vitro-differentiated cardiomyocytes. In some embodiments, a population of cells comprises at least 100 cells, at least 500 cells, at least 1000 cells, at least 1×10⁴ cells, at least 1×10⁵ cells, at least 1×10⁶ cells, at least 1×10⁷ cells, at least 1×10⁸ cells, at least 1×10⁹ cells, at least 1×10¹⁰ cells, at least 1×10¹¹ cells, at least 1×10¹² cells, at least 1×10¹³ cells, at least 1×10¹⁴ cells, at least 1×10¹⁵ cells, or more.

Confirmation of cardiomyocyte differentiation and maturation can be assessed by assaying sarcomere morphology and structural characterization of actin and myosin. The structure of cardiac sarcomeres is highly ordered, thus one with ordinary skill in the art can recognize these proteins (actin, myosin, alpha-actinin, titin) and their arrangement in tissues or collections of cultured cells can be used as markers to identify mature muscle cells and tissues. Developing cardiac cells undergo “sarcomerogenesis,” which creates new sarcomere units within the cell. The degree of sarcomere organization provides a measure of cardiomyocyte maturity.

Immunofluorescence assays and electron microscopy for α-actinin, β-myosin, actin, cTnT, tropomyosin, and collagen, among others can be used to identify and measure sarcomere structures. Immunofluorescent images can be quantified for sarcomere alignment, pattern strength, and sarcomere length. This can be accomplished by staining the protein within the sarcomeres (e.g., α-actinin) and qualitatively or quantitatively determining if the sarcomeres are aligned. For a quantitative measurement of sarcomere alignment, several methods can be employed such as using a scanning gradient and Fourier transform script to determine the position of the proteins within the sarcomeres. This is done by using each image taken by a microscope and camera for individual analysis. Using a directional derivative, the image gradient for each segment can be calculated to determine the local alignment of sarcomeres. The pattern strength can be determined by calculating the maximum peaks of one-dimensional Fourier transforms in the direction of the gradient. The lengths of sarcomeres can be calculated by measuring the intensity profiles of the sarcomeres along this same gradient direction.

Cellular morphology can be used to identify structurally mature stem cell-derived cardiomyocytes. Non-limiting examples of morphological and structural parameters include, but are not limited to, sarcomere length, Z-band width, binucleation percentages, nuclear eccentricity, cell area, and cell aspect ratio.

The cell activity and maturation can be determined by a number of parameters such as electrical maturity, metabolic maturity, or contractile maturity of a cardiomyocyte.

Mature cardiomyocytes have functional ion channels that permit the synchronization of cardiac muscle contraction. The electrical function of cardiomyocytes can be measured by a variety of methods. Non-limiting examples of such methods include whole cell patch clamp (manual or automated), multielectrode arrays, field potential stimulation, calcium imaging and optical mapping, among others. Cardiomyocytes can be electrically stimulated during whole cell current clamp or field potential recordings to produce an electrical and/or contractile response. Furthermore, cardiomyocytes can be genetically modified, for example, to express a channel rhodopsin that allows for optical stimulation of the cells.

Measurement of field potentials and biopotentials of cardiomyocytes can be used to determine their differentiation stage and cell maturity. Without limitations, the following parameters can be used to determine electrophysiological function of e.g., cardiomyocytes: change in field potential duration (FPD), quantification of FPD, beat frequency, beats per minute, upstroke velocity, resting membrane potential, amplitude of action potential, maximum diastolic potential, time constant of relaxation, action potential duration (APD) of 90% repolarization, interspike interval, change in beat interval, current density, activation and inactivation kinetics, among others.

Electrical maturity is determined by one or more of the following markers as compared to a reference level: increased gene expression of an ion channel gene, increased sodium current density, increased inwardly-rectifying potassium channel current density, decreased action potential frequency, decreased calcium wave frequency, and decreased field potential frequency.

During a disease state, the electrophysiological function of cardiomyocytes can be compromised. For example, cardiomyocytes that have prolonged FPD and APD when compared with normal stem cell-derived cardiomyocytes are typically an indication of arrhythmic potential.

Adult cardiomyocytes have been shown to have enhanced oxidative cellular metabolism compared with fetal cardiomyocytes marked by increased mitochondrial function and spare respiratory capacity. Metabolic assays can be used to determine the differentiation stage and cell maturity of the stem cell-derived cardiomyocytes as described herein. Non-limiting examples of metabolic assays include cellular bioenergetics assays (e.g., Seahorse Bioscience XF Extracellular Flux Analyzer), and oxygen consumption tests.

Specifically, cellular metabolism can be quantified by oxygen consumption rate (OCR), OCR trace during a fatty acid stress test, maximum change in OCR, maximum change in OCR after FCCP addition, and maximum respiratory capacity among other parameters.

Furthermore, a metabolic challenge or lactate enrichment assay can provide a measure of stem cell-derived cardiomyocyte maturity or a measure of the effects of various treatments of such cells. Most mammalian cells generally use glucose as their main energy source. However, cardiomyocytes are capable of energy production from different sources such as lactate or fatty acids. In some embodiments, lactate-supplemented and glucose-depleted culture medium, or the ability of cells to use lactate or fatty acids as an energy source is useful to identify mature cardiomyocytes and variations in their function.

Metabolic maturity of in vitro-differentiated cardiomyocytes is determined by one or more of the following markers as compared to a reference level: increased activity of mitochondrial function, increased fatty acid metabolism, increased oxygen consumption rate (OCR), increased phosphorylated ACC levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA.

Contractility of cardiomyocytes can be measured by optical tracking methods such as video analysis. In addition to optical tracking, impedimetric measurements can also be performed. For example, the cardiomyocytes described herein can have contractility or beat rate measurements determined by xCelligence™ real time cell analysis (Acea Biosciences, Inc., San Diego, Calif.).

A useful parameter to determine cardiomyocyte function is beat rate. The frequency of the contraction, beat rate, change in beat interval (ABI), or beat period, can be used to determine stem cell differentiation stage, stem cell-derived cardiomyocyte maturity, and the effects of a given treatment on such rate. Beat rate can be measured by optical tracking. The beat rate is typically elevated in fetal cardiomyocytes and is reduced as cardiomyocytes develop. Without limitations, contractile parameters can also include contractile force, contraction velocity, relaxation velocity, contraction angle distribution, or contraction anisotropic ratio.

Contractile maturity is determined by one or more of the following markers as compared to a reference level: increased beat frequency, increased contractile force, increased level or activity of α-myosin heavy chain (α-MHC), increased level or activity of sarcomeres, decreased circularity index, increased level or activity of troponin, increased level or activity of titin N2b, increased cell area, and increased aspect ratio.

Cardiac/Cardiomyocyte Grafts

In one aspect, described herein is a method of transplanting cardiomyocytes, e.g., in vitro-differentiated cardiomyocytes, the method comprising contacting a cardiac tissue with a human cardiomyocyte as described herein, a pharmaceutical composition described herein, a transplant composition described herein, or using a cardiac delivery device as described herein to deliver cardiomyocytes to a subject in need thereof.

As used herein, the term “transplanting” or “transplant” is used in the context of the placement of cells, e.g. cardiomyocytes, as described herein into a subject, by a method or route which results in at least partial localization of the introduced cells at a desired site, such as a site of injury or repair, such that a desired effect(s) is produced. The cells e.g. cardiomyocytes can be implanted directly or into the cardiac tissue of the recipient, e.g., at or near a site, or into cardiac tissue of a subject with a cardiac disease. As one of skill in the art will appreciate, long-term engraftment of the cardiomyocytes is desired as cardiomyocytes generally do not proliferate to an extent that the heart can heal from an acute injury comprising cell death. In some embodiments, the cells are optionally transplanted on or within a scaffold or biocompatible material that supports viability of the implanted cardiomyocytes, and/or, for example, assists with keeping administered cells in the desired location for engraftment or promotes integration with native cardiac cells in a subject. Preferably, the cardiomyocytes are human stem cell derived-cardiomyocytes or in vitro-differentiated cardiomyocytes as described herein.

Scaffold Compositions:

In one aspect, the cardiomyocytes described herein can be admixed with or cultured on a preparation that provides a scaffold or patterned substrate to support the cells. Such a scaffold or patterned substrate can provide a physical advantage in securing the cells in a given location, e.g., after implantation, as well as a biochemical advantage in providing, for example, extracellular cues for the further maturation or, e.g., maintenance of phenotype until the cells are established.

A scaffold is a structure, comprising a biocompatible material including but not limited to a gel, sheet, or lattice that can contain the cells in a desired location but permit the entry or diffusion of factors in the environment necessary for survival and function. A number of biocompatible polymers suitable for a scaffold are known in the art.

Biocompatible synthetic, natural, as well as semi-synthetic polymers, can be used for synthesizing polymeric particles that can be used as a scaffold material. In one embodiment, a scaffold or extracellular matrix composition useful in the methods and compositions described herein can comprise one or more of a synthetic hydrogel, hyaluronic acid, proteoglycan, collagen, fibronectin, vitronectin, and fibrin.

In general, for the practice of the methods described herein, it is preferable that a scaffold biodegrades such that the cardiomyocytes can be isolated from the polymer prior to implantation or such that the scaffold degrades over time in a subject and does not require removal. Thus, in one embodiment, the scaffold provides a temporary structure or matrix for growth and/or delivery of cardiomyocytes to a subject in need thereof. In some embodiments, the scaffold permits human cells to be grown in a shape suitable for transplantation or administration into a subject in need thereof, thereby permitting removal of the scaffold prior to implantation and reducing the risk of rejection or allergic response initiated by the scaffold itself.

Examples of polymers which can be used include natural and synthetic polymers, although synthetic polymers are preferred for reproducibility and controlled release kinetics. Synthetic polymers that can be used include biodegradable polymers such as poly(lactide) (PLA), poly(glycolic acid) (PGA), poly(lactide-co-glycolide) (PLGA), and other polyhydroxyacids, poly(caprolactone), polycarbonates, polyamides, polyanhydrides, polyphosphazene, polyamino acids, polyortho esters, polyacetals, polycyanoacrylates and biodegradable polyurethanes; non-biodegradable polymers such as polyacrylates, ethylene-vinyl acetate polymers and other acyl-substituted cellulose acetates and derivatives thereof; polyurethanes, polystyrenes, polyvinyl chloride, polyvinyl fluoride, poly(vinyl imidazole), chlorosulphonated polyolefins, and polyethylene oxide. Examples of biodegradable natural polymers include proteins such as albumin, collagen, fibrin, silk, synthetic polyamino acids and prolamines; polysaccharides such as alginate, heparin; and other naturally occurring biodegradable polymers of sugar units. Alternately, combinations of the aforementioned polymers can be used. In one aspect, a natural polymer that is not generally found in the extracellular matrix can be used.

PLA, PGA and PLA/PGA copolymers are particularly useful for forming biodegradable scaffolds. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. Methods of preparing polylactides are well documented in the patent literature. The following U.S. patents, the teachings of which are hereby incorporated by reference, describe in detail suitable polylactides, their properties and their preparation: U.S. Pat. No. 1,995,970 to Dorough; U.S. Pat. No. 2,703,316 to Schneider; U.S. Pat. No. 2,758,987 to Salzberg; U.S. Pat. No. 2,951,828 to Zeile; U.S. Pat. No. 2,676,945 to Higgins; and U.S. Pat. Nos. 2,683,136; 3,531,561 to Trehu.

PGA is a homopolymer of glycolic acid (hydroxyacetic acid). In the conversion of glycolic acid to poly (glycolic acid), glycolic acid is initially reacted with itself to form the cyclic ester glycolide, which in the presence of heat and a catalyst is converted to a high molecular weight linear-chain polymer. PGA polymers and their properties are described in more detail in Cyanamid Research Develops World's First Synthetic Absorbable Suture”, Chemistry and Industry, 905 (1970).

Fibers can be formed by melt-spinning, extrusion, casting, or other techniques well known in the polymer processing area. Preferred solvents, if used to remove a scaffold prior to implantation, are those which are completely removed by the processing or which are biocompatible in the amounts remaining after processing.

Polymers for use in a matrix should meet the mechanical and biochemical parameters necessary to provide adequate support for the cells with subsequent growth and proliferation. The polymers can be characterized with respect to mechanical properties such as tensile strength using an Instron tester, for polymer molecular weight by gel permeation chromatography (GPC), glass transition temperature by differential scanning calorimetry (DSC) and bond structure by infrared (IR) spectroscopy.

The substrate or scaffold can optionally be nanopatterned or micropatterned, for example, with grooves and ridges that permit or facilitate growth, arrangement or maturity of cardiac tissues on the scaffold. Scaffolds can be of any desired shape and can comprise a wide range of geometries that are useful for the methods described herein. A non-limiting list of shapes includes, for example, patches, hollow particles, tubes, sheets, cylinders, spheres, and fibers, among others. The shape or size of the scaffold should not substantially impede cell growth, cell differentiation, cell proliferation or any other cellular process, nor should the scaffold induce cell death via e.g., apoptosis or necrosis. In addition, care should be taken to ensure that the scaffold shape permits appropriate surface area for delivery of nutrients from the surrounding medium to cells in the population, such that cell viability is not impaired. The scaffold porosity can also be varied as desired by one of skill in the art.

In some embodiments, attachment of the cells to a polymer is enhanced by coating the polymers with compounds such as basement membrane components, fibronectin, agar, agarose, gelatin, gum arabic, collagens types I, II, III, IV, and V, laminin, glycosaminoglycans, polyvinyl alcohol, mixtures thereof, and other hydrophilic and peptide attachment materials known to those skilled in the art of cell culture or tissue engineering. Examples of a material for coating a polymeric scaffold include polyvinyl alcohol and collagen. As will be appreciated by one of skill in the art, Matrigel™ is not suitable for administration to a human subject, thus the compositions described herein do not include Matrigel™.

In some embodiments it can be desirable to add bioactive molecules/factors to the scaffold. A variety of bioactive molecules can be delivered using the matrices described herein.

In one embodiment, the bioactive factors include growth factors. Examples of growth factors include platelet derived growth factor (PDGF), transforming growth factor alpha or beta (TGFβ), bone morphogenic protein 4 (BMP4), fibroblastic growth factor 7 (FGF7), fibroblast growth factor 10 (FGF10), epidermal growth factor (EGF/TGFα), vascular endothelium growth factor (VEGF), some of which are also angiogenic factors. Amiodrone can also be added to a scaffold or matrix as described herein.

These factors are known to those skilled in the art and are available commercially or described in the literature. Bioactive molecules can be incorporated into the matrix and released over time by diffusion and/or degradation of the matrix, or they can be suspended with the cell suspension.

Combination Treatment

Also contemplated herein is the treatment or systemic delivery of other agents to a subject in combination with pre-treated cardiomyocytes as described herein, for example, as an adjunct agent. In some embodiments, such agents are not co-administered with the cardiomyocytes in the same composition nor are they used to pre-treat cardiomyocytes but rather are administered at a separate time or via a separate composition. In some embodiments, the agents to be administered in combination with pre-treated cardiomyocytes comprise one or more additional anti-arrhythmic agents selected from the group consisting of: ivabradine, lidocaine, flecainide, propafenone, sotalol and metoprolol. In one embodiment, ivabradine is administered by systemic delivery as an adjunct therapy to treat or prevent engraftment arrhythmia in a subject being treated with pre-treated cardiomyocytes as described herein. The combination therapy can be administered concurrently following administration of the pre-treated cardiomyocytes.

As used herein, the term “concurrently” is not limited to the administration of the two or more agents at exactly the same time, but rather, it is meant that they are administered to a subject in a sequence and within a time interval such that they can act together. For example, the combination of therapeutics can be administered at the same time or sequentially in any order at different points in time; however, if not administered at the same time, they should be administered sufficiently close in time so as to provide the desired therapeutic effect, preferably in a synergistic fashion. The agent can be administered separately from the pre-treated cardiomyocytes, in any appropriate form and by any suitable route. When the additional agent is administered in combination with the pre-treated cardiomyocytes, they need not be administered in the same pharmaceutical composition and it is understood that they can be administered in any order to a subject in need thereof. For example, the combination agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the pre-treated cardiomyocytes, to a subject in need thereof (or vice versa).

In some embodiments, the therapeutic agent used in combination with the pre-treated cardiomyocytes is more effective than would be seen with either agent alone. In some embodiments, delivery is such that the reduction in a symptom, or other parameter related to the disorder is greater than what would be observed with either therapeutic agent alone. The effect of such a combination can be partially additive, wholly additive, or greater than additive. The agent and/or other therapeutic agents, procedures or modalities can be administered during periods of active disease, or during a period of persistence or less active disease.

In certain embodiments, the administered amount or dosage of the anti-arrhythmic agent used to pre-treat cardiomyocytes when administered in combination with a second therapeutic agent (e.g., a second anti-arrhythmic agent such as ivadrabine) is lower (e.g., at least 20%, at least 30%, at least 40%, or at least 50%) than the amount or dosage of the first agent used to pre-treat the cardiomyocytes when administered alone.

Pharmaceutically Acceptable Carriers

The methods of administering human cardiomyocytes (e.g., pretreated cardiomyocytes) to a subject as described herein involve the use of therapeutic compositions comprising such cells. Therapeutic compositions contain a physiologically tolerable carrier together with the cell composition and optionally at least one additional bioactive agent, polypeptide(s), nucleic acid(s) encoding said polypeptide, or factor(s) as described herein, dissolved or dispersed therein as an active ingredient.

In a preferred embodiment, the therapeutic composition is not substantially immunogenic when administered to a mammal or human patient for therapeutic purposes, unless so desired. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset, transplant rejection, allergic reaction, and the like. A pharmaceutically acceptable carrier will not promote the raising of an immune response to an agent with which it is admixed, unless so desired. The preparation of a composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared.

A transplant composition for humans can include one or more pharmaceutically acceptable carrier or materials as excipients. In contrast, a cell culture composition (not for human transplant) typically will use research reagents like cell culture media as an excipient. Cardiomyocytes can also be administered in an FDA-approved matrix/scaffold or in combination with FDA-approved drugs as described above.

In general, the compositions comprising cardiomyocytes described herein are administered as suspension formulations where the cells are admixed with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the human cardiac progenitor cells as described herein using routine experimentation.

A cell composition can also be emulsified or presented as a liposome composition, provided that the emulsification procedure does not adversely affect cell viability. The cells and any other active ingredient can be mixed with excipients which are pharmaceutically acceptable and compatible with the active ingredient and in amounts suitable for use in the therapeutic methods described herein.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions as described herein that is effective in the treatment of a particular disorder or condition will depend on the nature of the disorder or condition, and can be determined by standard clinical techniques.

Cryopreservation:

In some embodiments, cardiomyocytes as described herein, including pre-treated cardiomyocytes, are cryopreserved, i.e., frozen for later thawing and administration. Cryopreservation and cryopreservatives are well known in the art, and include, for example, suspension of cells in medium containing DMSO (e.g., at or about 7.5-15%) or glycerol (e.g., at or about 10%), among other cryopreservatives. Cryopreservatives can be obtained from commercial sources and include e.g., Cryostor™ (StemCell Technologies, Vancouver, BC), CryoStor Dlite™ (BioLife Solutions, Inc. Bothell, Wash.) or trehalose (Millipore Sigma, St. Louis, Mo.), among others. Mammalian cells, including cardiomyocytes are generally frozen slowly, e.g., by reducing temperature about ⁻1° C. per minute, down to a temperature of −70°-⁻90° C. Storage can be at −80° C., e.g., in an ultra-low temperature freezer, or, for example, on dry ice or under liquid nitrogen.

Administration and Efficacy

Provided herein are methods for treating a cardiac disease, a cardiac disorder, a cardiac injury, heart failure, or myocardial infarction comprising administering cardiomyocytes to a subject in need thereof. Such administered cardiomyocytes can be pre-treated or co-administered with a given anti-arrhythmic agent (e.g., amiodarone). In some embodiments, methods and compositions are provided herein for the prevention of an anticipated disorder e.g., heart failure following myocardial injury.

Measured or measurable parameters include clinically detectable markers of disease, for example, elevated or depressed levels of a clinical or biological marker, as well as parameters related to a clinically accepted scale of symptoms or markers for a disease or disorder. It will be understood, however, that the total usage of the compositions and formulations as disclosed herein will be decided by the attending physician within the scope of sound medical judgment. The exact amount required will vary depending on factors such as the type of disease being treated.

The term “effective amount” as used herein refers to the amount of a population of cardiomyocytes needed to alleviate at least one or more symptoms of a disease or disorder, including but not limited to an injury, disease, or disorder. An “effective amount” relates to a sufficient amount of a composition to provide the desired effect, e.g., treat a subject having an infarct zone following myocardial infarction, improve cardiomyocyte engraftment, prevent onset of heart failure following cardiac injury, enhance vascularization of a graft, etc. The term “therapeutically effective amount” therefore refers to an amount of human cardiomyocytes or a composition such cells that is sufficient to promote a particular effect when administered to a typical subject, such as one who has, or is at risk for, a cardiac disease or disorder. In certain embodiment, the cardiomyocytes are delivered with a therapeutically effective amount of an anti-arrhytmic agent to prevent the onset of, or reduce the impact of an an engraftment arrhythmia. An effective amount as used herein would also include an amount sufficient to prevent or delay the development of a symptom of the disease, alter the course of a disease symptom (for example but not limited to, slow the progression of a symptom of the disease), or reverse a symptom of the disease. It is understood that for any given case, an appropriate “effective amount” can be determined by one of ordinary skill in the art using routine experimentation.

In some embodiments, the subject is first diagnosed as having a disease or disorder affecting the myocardium prior to administering the cells according to the methods described herein. In some embodiments, the subject is first diagnosed as being at risk of developing a disease (e.g., heart failure following myocardial injury) or disorder prior to administering the cells.

One of skill in the art can determine the number of cardiomyocytes needed for a graft. In some embodiments, about 10 million cardiomyocytes to about 10 billion cardiomyocytes are administered to the subject. For use in the various aspects described herein, an effective amount of human cardiomyocytes comprises at least 1×10³, at least 1×10⁴, at least 1×10⁵, at least 5×10⁵, at least 1×10⁶, at least 2×10⁶, at least 3×10⁶, at least 4×10⁶, at least 5×10⁶, at least 6×10⁶, at least 7×10⁶, at least 8×10⁶, at least 9×10⁶, at least 1×10⁷, at least 1.1×10⁷, at least 1.2×10⁷, at least 1.3×10⁷, at least 1.4×10⁷, at least 1.5×10⁷, at least 1.6×10⁷, at least 1.7×10⁷, at least 1.8×10⁷, at least 1.9×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 5×10⁸, at least 7×10⁸, at least 1×10⁹, at least 2×10⁹, at least 3×10⁹, at least 4×10⁹, at least 5×10⁹ or more cardiomyocytes.

In some embodiments, an effective amount of cardiomyocytes is administered to a subject by intracardiac administration or delivery. As defined herein, “intracardiac” administration or delivery refers to all routes of administration whereby a population of cardiomyocytes is administered in a way that results in direct contact of these cells with the myocardium of a subject, including, but not limited to, direct cardiac injection, intra-myocardial injection(s), intra-infarct zone injection, injection during surgery (e.g., cardiac bypass surgery, during implantation of a cardiac mini-pump or a pacemaker, etc.). In some such embodiments, the cells are injected into the myocardium (e.g., cardiomyocytes), or into the cavity of the atria and/or ventricles. In some embodiments, intracardiac delivery of cells includes administration methods whereby cells are administered, for example as a cell suspension, to a subject undergoing surgery via a single injection or multiple “mini” injections into the desired region of the heart.

The choice of formulation will depend upon the specific composition used and the number of cardiomyocytes to be administered; such formulations can be adjusted by the skilled practitioner. However, as an example, where the composition is cardiomyocytes in a pharmaceutically acceptable carrier, the composition can be a suspension of the cells in an appropriate buffer (e.g., saline buffer) at an effective concentration of cells per mL of solution. The formulation can also include cell nutrients, a simple sugar (e.g., for osmotic pressure regulation) or other components to maintain the viability of the cells. Alternatively, the formulation can comprise a scaffold, such as a biodegradable scaffold.

In some embodiments, additional agents to aid in treatment of the subject can be administered before or following treatment with the cardiomyocytes as described. Such additional agents can be used to prepare the target tissue for administration of the progenitor cells. Alternatively, the additional agents can be administered after the cardiomyocytes to support the engraftment and growth of the administered cell into the heart, or other desired administration site. In some embodiments, the additional agent comprises growth factors, such as VEGF or PDGF. Other exemplary agents can be used to reduce the load on the heart while the cardiomyocytes are engrafting (e.g., beta blockers, medications to lower blood pressure etc.).

The efficacy of treatment can be determined by the skilled clinician. However, a treatment is considered “effective treatment,” as the term is used herein, if any one or all of the symptoms, or other clinically accepted symptoms or markers of disease, e.g., cardiac disease, heart failure, cardiac injury and/or a cardiac disorder are reduced, e.g., by at least 10% following treatment with a composition comprising human cardiomyocytes as described herein. Methods of measuring these indicators are known to those of skill in the art and/or described herein. In one embodiment, treatment is effective if transplanted cardiomyocytes engraft without substantially causing engraftment arrhythmia as described herein. By “without substantially causing” in this context is meant that engraftment arrhythmia does not occur, or that any disturbances in rate or rhythm caused by the introduction of cardiomyocytes as described herein is at least 20% less in duration and/or severity, including at least 30%, at least 40%, at least 50%, at least 60%, at least 70%, at least 80%, at least 90% less relative to the engraftment of analogous cardiomyocytes that are not treated or modified as described herein.

In some embodiments, efficacious treatment using pre-treated cardiomycocytes can be assessed by measuring a decrease in arrhythmia duration, decrease in automaticity of the engrafted cells, a decrease in arrhythmia buden, decrease in peak heart rate, restoration of a normal sinus rhythm, or reduction of number of tachycardia episodes. For example, the arrhythmia burden of a subject administered pre-treated cardiomyocytes as described herein is at least 10% lower than the arrhythmia burden in a substantially similar subject treated with cardiomyocytes that were not pre-treated with an anti-arrhythmia agent or lower than the expected arrhythmia burden of the subject treated with cells that were not pre-treated with anti-arrhythmia agents. In some embodiments, the arrhythmia burden is reduced by at least 20%, at least 25%, at least 30%, at least 40%, at least 50%, at least 60%, at least 70%, at least 75%, at least 80%, at least 85%, at least 90%, at least 95%, at least 98%, at least 99% or even 100% with pre-treated cardiomyocytes as compared to non-pre-treated cardiomyocytes.

In some embodiments, the peak heart rate of subjects administered pre-treated cardiomyocytes is reduced by at least 5 bpm, at least 10 bpm, at least 15 bpm, at least 20 bpm, at least 25 bpm, at least 30 bpm, at least 35 bpm, at least 40 bpm, at least 45 bpm, at least 50 bpm, at least 60 bpm, at least 70 bpm, at least 80 bpm, at least 90 bpm, at least 100 bpm or more compared to the peak heart rate of subjects administered non-pre-treated cardiomyocytes.

In some embodiments, subjects having pre-treated cardiomyocytes have a delayed onset of arrhythmia by at least 1 day, 2 days, 3 days, 4 days, 5 days, 6 days, 7 days, 10 days, 12 days, 14 days, 3 weeks or 4 weeks or more compared to subjects treated with non-pre-treated cardiomyocytes.

Indicators of a cardiac disease or cardiac disorder, or cardiac injury include functional indicators or parameters, e.g., stroke volume, heart rate, left ventricular ejection fraction, heart rate, heart rhythm, blood pressure, heart volume, regurgitation, etc. as well as biochemical indicators, such as a decrease in markers of cardiac injury, such as serum lactate dehydrogenase, or serum troponin, among others. As one example, myocardial ischemia and reperfusion are associated with reduced cardiac function. Subjects that have suffered an ischemic cardiac event and/or that have received reperfusion therapy have reduced cardiac function when compared to that before ischemia and/or reperfusion. Measures of cardiac function include, for example, ejection fraction and fractional shortening. Ejection fraction is the fraction of blood pumped out of a ventricle with each heartbeat. The term ejection fraction applies to both the right and left ventricles. LVEF refers to the left ventricular ejection fraction (LVEF). Fractional shortening refers to the difference between end-diastolic and end-systolic dimensions divided by end-diastolic dimension.

Non-limiting examples of clinical tests that can be used to assess cardiac functional parameters include echocardiography (with or without Doppler flow imaging), electrocardiogram (EKG), exercise stress test, Holter monitoring, or measurement of β-natriuretic peptide.

Where necessary or desired, animal models of injury or disease can be used to gauge the effectiveness of a particular composition as described herein. For example, an isolated working rabbit or rat heart model, or a coronary ligation model in either canines or porcines can be used. Animal models of cardiac function are useful for monitoring infarct zones, coronary perfusion, electrical conduction, left ventricular end diastolic pressure, left ventricular ejection fraction, heart rate, blood pressure, degree of hypertrophy, diastolic relaxation function, cardiac output, heart rate variability, and ventricular wall thickness, etc. The porcine model described in the examples herein is particularly preferred.

In some embodiments, a composition comprising the cardiomyocytes as described herein is delivered at least 6 hours following the initiation of reperfusion, for example, following a myocardial infarction. During an ischemic insult and subsequent reperfusion, the microenvironment of the heart or that of the infarcted zone can be too “hostile” to permit engraftment of cardiomyocytes administered to the heart. Thus, in some embodiments it is preferable to administer such compositions at least 6 hours, at least 12 hours, at least 18 hours, at least 24 hours, at least 36 hours, at least 48 hours, at least 60 hours, at least 72 hours, at least 84 hours, at least 96 hours, at least 5 days, at least 6 days, at least 7 days, at least 8 days, at least 9 days, at least 10 days or more following the initiation of reperfusion. In some embodiments, the compositions comprising cardiomyocytes as described herein can be administered to an infarcted zone, peri-infarcted zone, ischemic zone, penumbra, or the border zone of the heart at any length of time after a myocardial infarction (e.g., at least 1 month, at least 6 months, at least one year, at least 2 years, at least 5 years, at least 10 years, at least 20 years, at least 30 years or more), however as will be appreciated by those of skill in the art, the success of engraftment following a lengthy interval of time after infarct will depend on a number of factors, including but not limited to, amount of scar tissue deposition, density of scar tissue, size of the infarcted zone, degree of vascularization surrounding the infarcted zone, etc. As such, earlier intervention by administration of compositions comprising cardiomyocytes may be more efficacious than administration after e.g., a month or more after infarct.

Compositions comprising cardiomyocytes as described herein can be administered to any desired region of the heart including, but not limited to, an infarcted zone, peri-infarcted zone, ischemic zone, penumbra, the border zone, areas of wall thinning, areas of non-compaction, or in area(s) at risk of maladaptive cardiac remodeling.

### EXAMPLES

### Example 1: Prevention of Engraftment Arrhythmia with Antiarrhythmic Agents

Myocardial infarction (MI) remains the leading cause of heart failure and death in the United States and around the world (1). During MI, approximately one billion cardiomyocytes are permanently lost, often leading to debilitating heart failure. Current treatments can slow the initiation and progression of heart failure, but none replaces lost myocardium, short of orthotopic heart transplantation, which remains restricted in availability and indication (2). Human pluripotent stem cells (hPSCs, comprising embryonic stem cells [ESCs] and their reprogrammed cousins, induced pluripotent stem cells [iPSCs]) are a renewable source of cardiomyocytes (CMs). Transplantation of hPSC-derived cardiomyocytes (hPSC-CMs) into infarcted myocardium of small animals—mice, rats, guinea pigs—has shown stable engraftment (3-7). More recently, remuscularization and functional benefit has been shown in infarcted non-human primates (NHP) following transplantation of human pluripotent stem cell hPSC-CM (8-10). In addition to functional remuscularization, the human graft vascularizes and electromechanically couples with the host myocardium within one-month post-transplant and remains durable up to three months, the longest time tested.

Although no arrhythmias were observed in smaller animals, researchers consistently observe ventricular arrhythmias following hPSC-CM transplantation in NHPs (8-10) and pigs (11) which are termed ‘engraftment arrhythmias (EAs)’. EAs are generally transient, occurring within a week of transplantation and generally resolving spontaneously after one month. Based on electrical mapping, overdrive pacing, and cardioversion studies, EAs appear to originate focally in the graft or peri-graft myocardium and function as automatous foci rather than reentrant pathways (9,11). Although EA is reasonably well tolerated in NHPs, the Laflamme group (11) reported that EA can be lethal in some pigs. For this reason, EA has emerged as the biggest impediment to clinical translation of human cardiomyocyte transplantation (12).

It is hypothesized that the risk of EA may be mitigated by treatment with clinically available anti-arrhythmic drugs. Because the pig shows heightened sensitivity to EAs, and because it is a well-established model in cardiovascular research (13) and cell therapy (14) whose larger size permits use of percutaneous delivery catheters, the inventors chose to test the hypothesis in this large animal model. In the first phase of the study, a panel of anti-arrhythmic agents was screened. Amiodarone and ivabradine emerged independently as the most promising agents for control of rhythm and rate, respectively. A second phase was then performed to test the effect of combined amiodarone and ivabradine treatment. It was found that this regimen reduced sudden cardiac death, as well as suppressed tachycardia and arrhythmia.

Methods and Materials

hESC-CM Production

These studies were approved by the University of Washington Stem Cell Research Oversight Committee. Two lines of hESCs were used in this study. Initial subjects received H7 (WiCell)-derived cardiomyocytes that were cultured, expanded, and differentiated in suspension-culture format by collaborators at the Center for Applied Technology Development at the City of Hope in California as previously described (8,9,15). The majority of subjects received RUES2 (Rockefeller University)-derived cardiomyocytes in stirred suspension culture format. Briefly, RUES2 hESC were cultured to form aggregates and were expanded in commercially available media (Gibco Essential 8). For cardiac differentiation, suspension adapted pluripotent aggregates were induced to differentiate in RPMI-1640 (Gibco), MCDB-131 (Gibco), or M199 (Gibco) supplemented with B-27 (Gibco) or serum albumin by timed use of small molecule GSK 3 inhibitors and Wnt/β-catenin signal pathway inhibitors (Tocris). Twenty-four hours prior to cryopreservation, RUES2 hESC-CMs were heat-shocked to enhance their survival after harvest, cryopreservation, thaw, and transplantation.

Cardiomyocyte aggregates were dissociated by treatment with Liberase™ TH (Fisher) and TrypLE™ (Gibco) and were cryopreserved in CryoStor™ CS10 (Stem Cell Technologies) supplemented with 10 μM Y-27632 (Stem Cell Technologies) using a controlled-rate liquid nitrogen freezer. Approximately 3 h before transplantation, cryopreserved hESC-CM were removed from cryogenic storage (−150° C. to −196° C.) and thawed in a 37° C. water bath (2 min±30 s). RPMI-1640 supplemented with B-27 and ≥200 Kunitz Units/mL DNase™ I (Millipore) was added to the cell suspension to dilute the cryopreservation media. Subsequent wash steps were done using RPMI-1640 basal media in progressively smaller volumes in order to concentrate the cell suspension. For the last centrifugation step, the cell pellet was resuspended in a sufficient volume of RPMI-1640 to achieve a target cell density for injection of ˜0.3×10⁹ cells/mL in 1.6 mL. The final volume of the cell suspension was determined by the results of a count sampled before the final centrifugation step. Cell counts were performed as described previously to achieve a final total dose of 500×10⁶ live cells (9).

Study Design

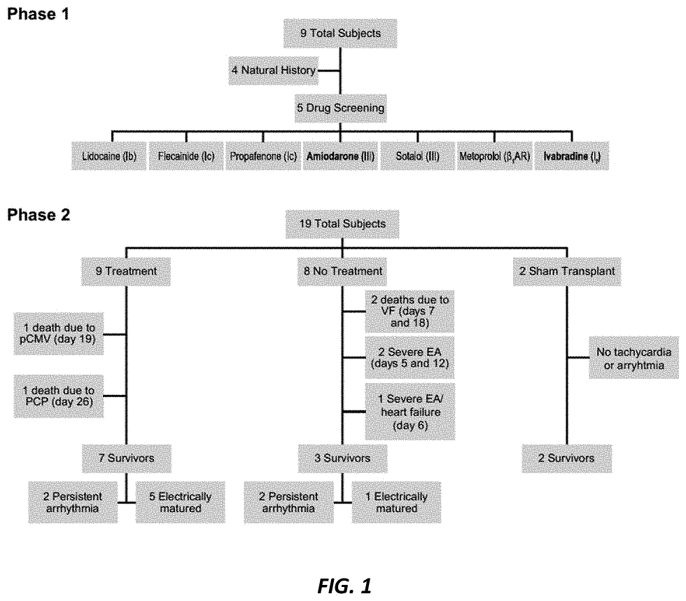

The objective of this study was to identify a pharmacological regimen to attenuate arrhythmias following cardiac remuscularization therapy. This study was designed in two phases: the first, to observe the natural history of EA and screen various antiarrhythmic agents for efficacy, and the second, to test for efficacy (FIG. 1). All subjects were 30-40 kg castrated male Yucatan minipigs between 6-13 months of age (S&S Farms). In Phase 1, nine subjects underwent cardiac remuscularization therapy with 500×10⁶ hESC-CM delivered by direct surgical transepicardial injections or later, by percutaneous transendocardial injections. The first four subjects (one non-infarcted and three infarcted) were followed to learn the natural history of EA and establish clinical endpoints and parameters for the Phase 2 drug trial. The subsequent five subjects underwent systematic dosing with antiarrhythmic agents with continuous electrocardiography (ECG) monitoring to determine effect on rhythm and rate (Table 1).

TABLE 1

| Drug | Dosage | Effect |
|---|---|---|
| Lidocaine | 100 mg IV | Modest HR effect, rare |
| (Ib) | | cardioversion |
| Flecainide | 2 mg/kg PO, 4 mg/kg PO, | No response on HR or EA |
| (Ic) | 6 mg/kg PO, | burden |
| | 10 mg/kg PO | |
| Propafenone | 1 mg/kg IV, 2 mg/kg IV, | No response on HR, transient |
| (Ic) | 3 mg/kg IV | cardioversion* |
| Amiodarone | 150 mg IV | Modest HR effect, frequent |
| (III) | | cardioversion |
| Sotalol (III) | 1 mg/kg PO, 2 mg/kg PO, | No response on HR or EA |
| | 4 mg/kg PO | burden |
| Metoprolol | 5 mg IV, 25 mg PO BID, | Moderate HR effect (IV only), |
| (β₁AR) | 50 mg PO BID, | no response on EA burden |
| | 75 mg PO BID | |
| Ivabradine | 2.5 mg PO, 5 mg PO BID, | Robust dose-dependent HR |
| (I_{f}) | 10 mg PO BID, 15 mg BID; | effect (PO only)**, no |
| | 1 mg/kg IV, 2 mg/kg IV | response on EA burden |

*Severe nausea/emesis observed at therapeutic doses, limiting clinical utility
**Severe bradycardia
Abbreviations:
β₁AR, β1-adrenergic receptor;
BID, twice daily;
HR, heart rate;
EA, engraftment
arrhythmia;
I_{f}, funny current;
PO, oral;
RyR₁, ryanodine receptor 1;
VT, ventricular tachycardia

Among the nine subjects in Phase 1, the inventors observed high mortality, with six out of nine experiencing ventricular fibrillation (VF) or tachycardia-induced heart failure requiring euthanasia. VF typically followed frequent episodes of unstable EA>350 beats per minute (bpm), and tachycardia-induced heart failure requiring euthanasia was characterized by chronically elevated heart rates >150 bpm. Based on promising results from Phase 1, the inventors proceeded to Phase 2, a prospective drug trial to prevent EA-related mortality.

In Phase 2, a two-drug antiarrhythmic study was performed with amiodarone and ivabradine and an additional 17 subjects (9 treated, 8 untreated) were enrolled, who underwent MI and percutaneous hESC-CM remuscularization at two weeks post-MI. Two additional subjects underwent MI with sham vehicle injection to serve as sham transplant controls (FIG. 1, FIG. 7). The primary endpoint was prespecified as combined cardiac death (either spontaneous death from arrhythmia or heart failure, or clinically directed euthanasia necessitated by tachycardia >350 bpm or signs of heart failure).

Prespecified secondary endpoints were suppression of tachycardia, percent time in arrhythmia (arrhythmia burden) and resolution of arrhythmia, termed electrical maturation and defined as arrhythmia burden <25% for 48 consecutive hours. Antiarrhythmic therapy was discontinued after electrical maturation or post-transplantation day 30, whichever was earlier. To prevent tachycardia-induced cardiomyopathy, ivabradine treatment was titrated to maintain a target heart rate of <150 bpm. Based on early experience that tachycardia >350 bpm often degenerated to VF, subjects were euthanized humanely if heart rates >350 bpm were reached. Continuous telemetric ECG was monitored for eight weeks total (two weeks post-MI and six weeks post-transplantation). Of note, subjects 1 and 2 (no treatment) and 3 and 4 (treatment) received H7 hESC-CM and subjects 1, 2 and 3 were transplanted surgically prior to adopting percutaneous delivery. Subject 5 was euthanized on day 37 as a prespecified endpoint following electrical maturation, prior to extending the study duration to 6 weeks post-transplantation for extended treatment washout and monitoring.

Animal Care

All protocols were approved and conducted in accordance with the University of Washington (UW) Office of Animal Welfare and the Institutional Animal Care and Use Committee. Animals received ad libitum water and were fed twice a day (Lab Diet-5084 Laboratory Porcine Grower Diet). For surgical procedures, anesthesia was induced with a combination of intramuscular butorphanol, acepromazine and ketamine. Animals were intubated and mechanically ventilated using isoflurane and oxygen to maintain a surgical plane of anesthesia. Vital signs were monitored continuously throughout each procedure. All animals received subcutaneous Buprenorphine SR-Lab (ZooPharm) for post-operative analgesia and were euthanized by intravenous Euthasol (Virbac). All post-mortem examinations were performed by a blinded board-certified veterinary pathologist.

Porcine Myocardial Infarction Model

Percutaneous ischemia/reperfusion injury was induced as previously described in NHP (9) with modification for the porcine model. A 5-8 cm incision was made in the femoral triangle and the femoral artery was exposed by blunt dissection. Prior to obtaining vascular access, heparin was administered to achieve therapeutic anticoagulation (activated coagulation time >250 sec). A 5-French guidewire/introducer sheath system (Terumo Medical) was placed into the femoral artery and secured. Continuous ECG, invasive arterial blood pressure, pulse-oximetry and capnography were monitored throughout the procedure. Intravenous amiodarone 150 mg and lidocaine 100 mg were administered as single boluses prior to ischemia to minimize the risk of arrhythmia. Under fluoroscopic guidance (OEC 9800 Plus, GE Medical Systems), a 5-French Judkins right 2 or hockey stick guide catheter (Boston Scientific) was advanced into the ascending aorta to selectively engage the ostium of the left main coronary artery. Coronary angiography was performed using hand injections of contrast (Visipaque) and a 0.035″ coronary guidewire (Runthrough NS Extra Floppy, Terumo Medical) was placed into the distal left anterior descending coronary artery (LAD). An appropriately sized angioplasty balloon catheter was then positioned into the mid-LAD distal to the first diagonal branch artery and inflated to the minimum pressure required for total obstruction of distal perfusion as confirmed by angiography. Ischemia was confirmed by ST-segment elevation on the ECG. Animals were maintained under anesthesia with ventilatory and hemodynamic support for 90 minutes, after which the balloon was deflated to restore distal perfusion, again confirmed by fluoroscopy and ECG. The animal was observed for reperfusion arrhythmias and externally cardioverted if ventricular fibrillation occurred. Prior to recovery, all subjects received implantable telemetry units and central venous catheter placement. Briefly, the external jugular vein in the jugular furrow was exposed and a 5-French central venous catheter (Access Technologies) was inserted and tunneled out to the dorsal prescapular area. The telemetry transmitter (EMKA easyTEL+) was implanted in a subcutaneous pocket using the same incision in the jugular furrow, and subcutaneous leads were tunneled to capture the cardiac apex to base. The overall procedural mortality including the infarct was <10%.

Cardiac Remuscularization Therapy

Cell transplantation for three initial subjects (1-3) was performed by direct transepicardial injection into the peri-infarction region as previously described for NHP with minor modification (9). Briefly, a partial median sternotomy was performed to expose the infarcted anterior left ventricle. Purse-string sutures were preplaced at five discrete locations subtended by the LAD, targeting the central ischemic region and lateral border zones. After cinching the purse-string tightly around the needle, three injections of 100 μL each were performed by partial withdrawal and lateral repositioning, for a total of 15 injections to deliver total dose of 500×10⁶ hESC-CMs. All subsequent subjects (4-19) received cell transplantation via percutaneous transendocardial injection using the NOGA-MyoStar platform (BioSense Webster) to first map the infarct region in the left ventricle, and then to deliver 16 discrete endocardial injections of 100 each for total dose of 500×10⁶ hESC-CMs. Injections were only performed with excellent location and loop stability, ST-segment elevation and presence of premature ventricular contraction (PVC) with needle insertion in an appropriate location by electroanatomical map and unipolar volage. For both surgical and percutaneous cell transplantation, two-thirds of injections were placed into the peri-infarct border zone defined visually or by unipolar voltage of 5-7.5 mV and the remaining one-third into the central ischemic region defined visually or as unipolar voltage of <5 mV. Two subjects (9 and 10) were infarcted as per protocol but received sham injections of RMI-1640 vehicle without cells to serve as sham transplant controls.

Immunosuppression Therapy

All subjects received a three-drug immunosuppression regimen to prevent xenograft rejection as previously described with modification (9). For the initial regimen (subjects 1-6), five days prior to cell transplantation, oral cyclosporine A was started to maintain serum trough level of >400 ng/ml (approximately 250-1000 mg twice daily) for duration of the study. Two days prior to transplantation, oral methylprednisolone was started at 3 mg/kg for two weeks then titrated down to 1.5 mg/kg for the remainder of the study. On the day of transplantation, Abatacept™ (CTLA4-Ig, Bristol-Myers Squibb) 12.5 mg/kg was administered intravenously and dosed every two weeks thereafter. Due to complications related to immunosuppression (principally porcine cytomegalovirus and Pneumocystis pneumonia), the cyclosporine A trough level was decreased to >300 ng/ml and the methylprednisolone reduced to 1.0 mg/kg for subjects 7-19 without histologic evidence of rejection. Prophylactic oral cephalexin was administered for all subjects to prevent infection of the indwelling central venous catheter. Prophylactic sulfamethoxazole/trimethoprim was added after subject 3 developed Pneumocystis pneumonia. Prophylactic valganciclovir and probiotics were added after activation of endogenous porcine cytomegalovirus was found in subject 6.

Antiarrhythmic Treatment

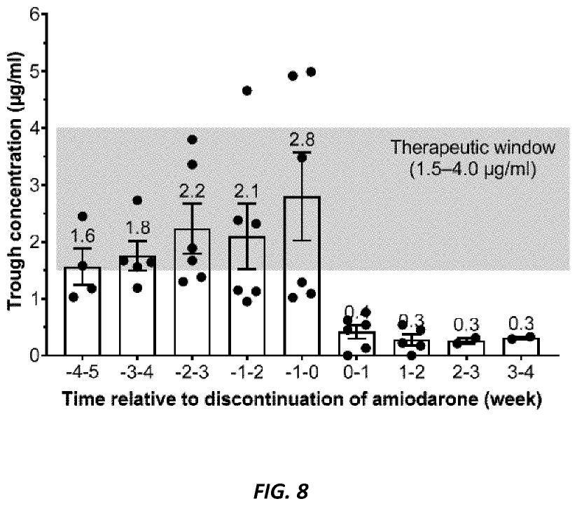

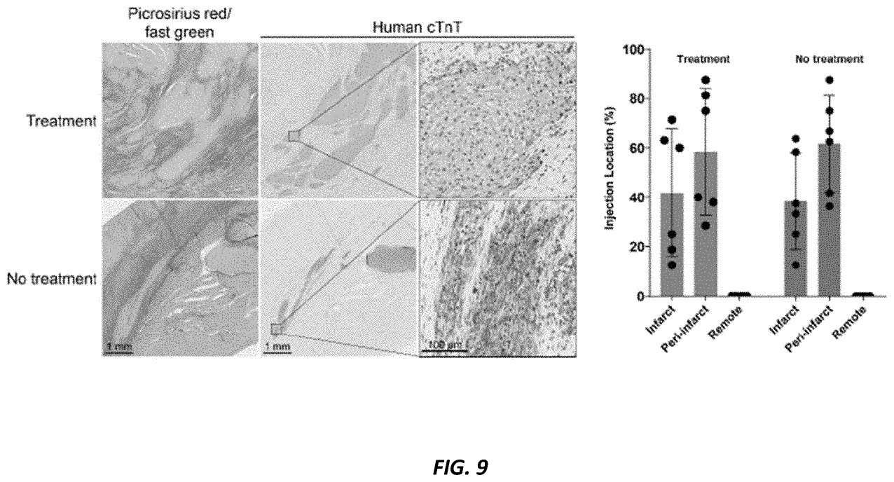

Treatment subjects were loaded with oral amiodarone 1000-1200 mg orally twice daily starting seven days prior to cell transplantation followed by maintenance dose of 400-1000 mg orally twice daily to maintain a steady-state plasma level of 1.5-4.0 μg/ml (FIG. 8). Ivabradine was started at 2.5 mg orally twice daily when sustained tachycardia reached >150 bpm and titrated every 3 days up to 15 mg twice daily for goal heart rate <125 bpm. All but one subject (subject 1) required adjunctive ivabradine for additional heart rate control. Antiarrhythmics were discontinued after electrical maturation was achieved or post-transplantation day 30, whichever was earlier, to allow for treatment washout and assess for recrudescence of arrhythmia. All subjects tolerated the antiarrhythmic regimen without complication. Untreated and sham transplant control subjects did not receive antiarrhythmic agents following the MI procedure, but otherwise received all immunosuppression and standard care.

Amiodarone Drug Monitoring

A novel liquid chromatography—mass spectrometry assay was established for amiodarone to monitor steady state serum levels in the porcine model and guide oral dosing to ensure efficacy and avoid dose-related toxicity. A target serum level of 1.5-4.0 μg/ml was extrapolated from prior human pharmacokinetic studies (16,17). Elimination kinetics after discontinuation of oral amiodarone therapy were also studied by obtaining weekly trough concentrations in 6 pigs (subjects 6, 7, 8, 13, 14, 16) (FIG. 8).

Electrocardiography (ECG) Analysis

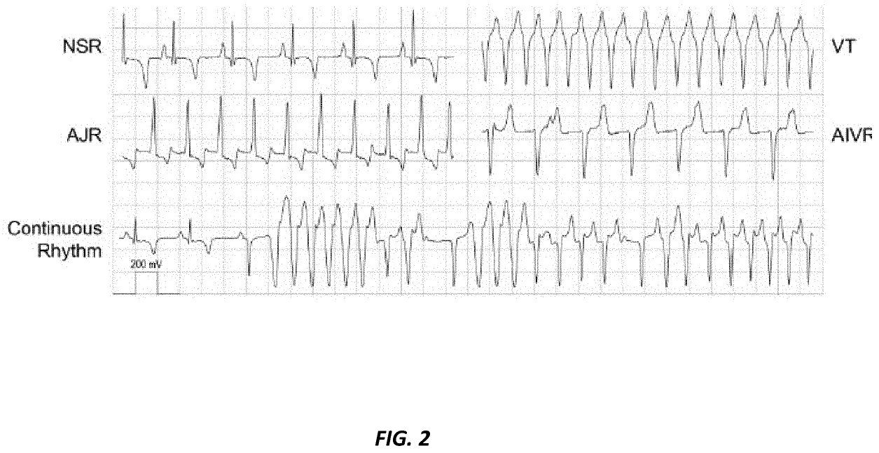

Telemetric ECG was continuously monitored in real-time from the time of myocardial infarction to detect the primary endpoint of cardiac death or unstable EA. Automated quantification of heart rate and arrhythmia burden was performed offline by a board-certified cardiologist using the ecgAUTO™ 3.3.5.10 software package (EMKA Technologies). Arrhythmia was defined as an ectopic beat (e.g. premature ventricular contraction) or rhythm (e.g. idioventricular rhythm, ventricular tachycardia). EA was typically observed as sustained and non-sustained ventricular tachyarrhythmia of varying rates and morphologies but also included slow and narrow complex ectopic rhythms (FIG. 2). Heart rate and arrhythmia burden were quantified for two continuous minutes every five minutes (40% of total rhythm was counted) and presented as daily averages.

Histologic Analysis

Histological studies were carried out as detailed previously with modification (8,9). Briefly, paraformaldehyde-fixed hearts were dissected to remove the atria and right ventricle before short-axis cross-sections were cut at 2.5 mm intervals. The weights of the whole heart, left ventricle and each slice were obtained before further partition into tissue cassettes. The tissue then was processed, embedded in paraffin, and 4 μm sections were cut for staining. For morphometry, infarct regions were identified by picrosirius red staining; human graft was identified by anti-human cardiac troponin T, stained using avidin-biotin reaction (ABC Kit, VectorLabs) followed by chromogenic detection via diaminobenzidine (Sigmafast, Sigma Life Science) (FIG. 9). The slides were digitized using a whole slide scanner (Nanozoomer, Hamamatsu), and the images were viewed and exported with NDP.view 2.6.13 (Hamamatsu). Areas of infarct and graft were analyzed using a custom-written algorithm in the ImageJ open source software platform (18). Briefly, after extracting images in TIFF format (19), the image foreground was segmented by a threshold derived from the distribution in brightness of its pixels, resulting in a binary mask that delineates the imaged tissue section. Subsequent color de-convolution by thresholding hue, brightness and saturation allowed segmentation of regions stained by Picro-Sirius Red stain or areas immunolabelled for human cardiac troponin-T. To separate scar from diffuse fibrosis, a cut-off for particle size was applied. Infarct size and graft size were calculated as the (percent area×block weight), summed for the entire ventricle, and expressed as a percentage of left ventricular mass or infarct mass, respectively.

Pilot Antiarrhythmic Screening in Pig

Five infarcted pigs underwent hESC-CM transplantation with all exhibiting stereotypic EA. Subjects were administered multiple trials antiarrhythmics and observed for acute response by continuous ECG monitoring. Intravenous agents were delivered as a bolus dose over 2 minutes. Oral agents were administrated by direct observation in a minimum of apples, apple sauce or pumpkin puree with daily feeding and dosed daily for dose escalation. A washout period of at least three days was provided between agents. Amiodarone was administered as the last agent for testing given concern for prolonged half-life and elimination kinetics. All agents were tested in at least two subjects.

Purkinje Fiber Histology

For thin sections, tissue was cut and trimmed to 1 cm×1 cm×3 mm, snap frozen in isopentane, and embedded in OCT™ (TissueTek). 10 μm sections were immersed in 100% methanol at −20° C. for 15 minutes and stained with standard immunofluorescence technique using stains described below. Images were acquired on a Leica SP8 confocal microscope.

For thick sections, 1 cm×1 cm×3 mm pieces of tissue containing graft were incubated in 100% methanol at 20° C. for 1 hour, rehydrated (80% methanol, 60% methanol, 0% methanol, diluted in PBS, 15-minute incubation at −20° C. for each reagent). 150 μm sections were cut on a Leica VT1200s vibratome and stained with standard immunofluorescence technique using stains described below.

Stained sections were then cleared using BABB as previously reported (34), and imaged on a Leica SP8 confocal microscope with 1 μm z-step increments.

Purkinje Fiber Staining

Sections were stained the following reagents: Hoechst 33342 (DNA, Thermo Fisher Scientific, #62249), Wheat germ agglutinin-Oregon Green (WGA, Thermo Fisher Scientific, #W6748), Phalloidin-647 (F-Actin, Thermo Fisher Scientific, #A22287), anti-Connexin 40 (Cx40, Alpha Diagnostics, #CXN40A), or anti-slow skeletal troponin I (ss-TnI, Novus, #NBP2-46170) with one of two anti-rabbit secondary antibodies (Alexa Fluor 555/647, Thermo Fisher Scientific, #A-31570/A-31573).

Statistical Analysis

Statistical analyses and graphing were performed using Prism 8.4.2 software (GraphPad) and Stata 15 (StataCorp, College Station, Tex.). Data are presented as mean±standard error of the mean (SEM). Comparisons were performed using Mann-Whitney test with significance threshold of P<0.05 unless otherwise specified. Error bar plots show how the mean±SEM of heart rate and arrhythmia burden varies over time in the two treatment groups. Kaplan-Meier plots show survival curves for the primary endpoint of cardiac death, unstable EA or heart failure, and for all-cause mortality. Cox proportional regression models are used to estimate the hazard ratio (HR) between the two treatment groups, for the primary outcome and for mortality. Significance is based on the likelihood ratio test and confidence intervals on HR are computed by inverting the likelihood test, based on varying the offset term in the stcox procedure in Stata.

Results

Percutaneous Delivery of hESC-CM in Infarcted Porcine Model

Catheter-based endocardial delivery of hESC-CM was safe and effective in remuscularizing the infarcted porcine heart (FIG. 9). No significant differences in myocardial infarct or cardiomyocyte graft sizes were observed between the treatment groups. The average infarct size for the treatment and no-treatment cohorts were comparable at 11.7±1.1% and 10.5±2.0% of the left ventricle, respectively (p=0.59). Graft size relative to infarct size was also comparable at 2.3±0.7% and 2.8±1.3% for treatment and no treatment, respectively (p=0.74). Delivery of hESC-CM successfully targeted the peri-infarct border zone and central ischemic regions as intended and resulted in discrete hPSC-CM grafts transplanted into host myocardium as previously reported (8-11). All grafts were located in the anterior, antero-septal and antero-lateral walls and appeared structurally immature at early time points before 2 weeks post-transplantation with increasing maturity up to the end of study as previously reported in pig (11).

Clinical History of Engraftment Arrhythmia

A flow chart for all animals in the study is shown in FIG. 1. No significant arrhythmias were noted in the two sham transplant subjects (9 and 10) that underwent myocardial infarction and percutaneous intracardiac injection of vehicle. All subjects that received human cardiomyocyte grafts developed EA between 2-6 days following cell transplantation. Initiation of EA was characterized by salvos of non-sustained VT, and this typically progressed to periods of sustained VT with rates ranging from 110 to 250 bpm (FIG. 2). The VT was often polymorphic, with the same animal showing different electrical axes and both wide- and narrow-complex tachycardia at different times. In 4 of the 8 untreated animals, EA was either fatal or necessitated euthanasia due to a prespecified endpoint of unstable tachycardia (defined as sustained heart rate >350 bpm). In one additional untreated case (subject 12), acute heart failure was noted clinically shortly after initiation of EA at a rate of 300 bpm, and based on recommendations from veterinary staff, the subject was euthanized. Signs of heart failure were subsequently confirmed on necropsy. In all other cases, EA was noted with a rapid acceleration to >350 bpm (subjects 11 and 12) and, in two cases, deterioration to VF prior to euthanasia (subjects 1 and 2) (Table 1). Three out of four arrhythmic endpoints occurred within the first three days of developing EA, and they occurred when tachyarrhythmia was nearly constant. Mean heart rate peaked at day 8 post-transplantation and began to decline after that, whereas the arrhythmia burden plateaued from days 8-16 and began to normalize thereafter. Of the three survivors in the untreated cohort, two did not normalized rhythm and experienced on average 42% arrythmia burden at the end of study (subjects 15 and 17). The single subject in the untreated cohort that normalized heart rate and rhythm did so on day post-transplant day 26 (subject 11).

Screening Drugs for Anti-Arrhythmic Effects

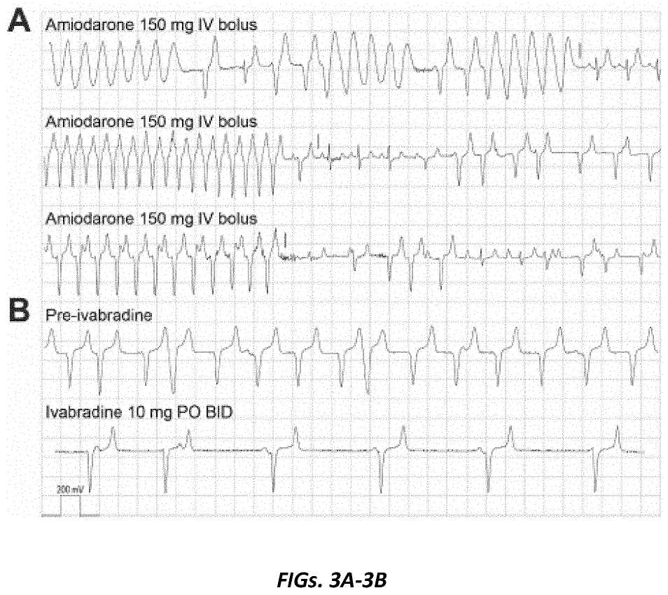

In Phase 1 of the study, the inventors screened six canonical antiarrhythmic agents broadly targeting sodium channels, potassium channels, and beta-adrenergic receptors: lidocaine (Ib, sodium channel inhibitor), flecainide (Ic, sodium channel inhibitor), propafenone (Ic, sodium channel inhibitor), amiodarone (III, potassium channel inhibitor), sotalol (III, potassium channel inhibitor) and metoprolol (β1-adrenergic receptor inhibitor) for effect on EA heart rate and rhythm. In addition, the funny current/HCN4 channel antagonist, ivabradine, was tested (Table 1). This series were not meant to be definitive but rather to rapidly identify the candidate agents. Animals were brought into the laboratory while in EA, anesthetized, and the effects of short-term intravenous infusion or oral treatment of anti-arrhythmic agents were studied. In three instances, intravenous amiodarone successfully cardioverted unstable EA from >350 bpm to a lower heart rate, typically including brief episodes of sinus rhythm (FIG. 3A). Oral ivabradine demonstrated robust dose-dependent effects on heart rate, but it did not restore sinus rhythm (FIG. 3B). Five of the other drugs had no significant effect in this screen (lidocaine, flecainide, sotalol, and metoprolol). Propafenone briefly reduced heart rate and restored sinus rhythm in two drug challenges, but this drug was associated with substantial gastrointestinal toxicity and not studied further (data not shown).

Amiodarone-Ivabradine Enhance Survival

Given their distinct mechanisms of action and complementary effects on heart rate and rhythm, the inventors formally tested the hypothesis that chronic amiodarone with adjunctive ivabradine would reduce a combined primary endpoint of cardiac death, unstable EA>350 bpm and heart failure in Phase 2 of the study. A total of nine treated, eight untreated, and two sham transplant subjects were enrolled in the study with similar baseline and cell transplantation characteristics (Table 2).

TABLE 2

| | | | | | | | | Infarct | Graft | | | Arrhythmia burden - | | |
|---|---|---|---|---|---|---|---|---|---|---|---|---|---|---|
| | | | | Cell | | | | size - | size - % | HR - bpm | | % of day | | |
| Subject | Age - mo | Weight - kg | MI | line | Approach | CTnT - % | Viability - % | % LV | Infarct | Day 7 | Day 30 | Day 7 | Day 30 | Outcome |

Treatment

| | | | | | | | | | | | | | | |
|---|---|---|---|---|---|---|---|---|---|---|---|---|---|---|
| 3 | 7.7 | 34.7 | Yes | H7 | Surg | 98% | 89% | — | — | 72.8 | n/a | 2.0 | n/a | Euthanasia, day 26 |
| | | | | | | | | | | | | | | (PCP) |
| 4 | 7.6 | 32.0 | Yes | H7 | Perc | 98% | 89% | — | — | 92.7 | 93.9 | 86.0 | 97.8 | Survival |
| 5 | 7.9 | 33.0 | Yes | RUES2 | Perc | 91% | 89% | — | — | 162.6 | 80.9 | 25.6 | 0.5 | Survival |
| 6 | 7.7 | 33.4 | Yes | RUES2 | Perc | 91% | 90% | 10.1% | 1.4% | 99.6 | n/a | 47.1 | n/a | Euthanasia, day 19 |
| | | | | | | | | | | | | | | (pCMV) |
| 7 | 7.7 | 37.0 | Yes | RUES2 | Perc | 91% | 93% | 9.6% | 0.3% | 89.4 | 78.6 | 45.8 | 28.2 | Survival |
| 8 | 10.0 | 33.5 | Yes | RUES2 | Perc | 86% | 88% | 15.9% | 3.4% | 72.8 | 77.4 | 67.6 | 4.7 | Survival |
| 15 | 9.0 | 33.0 | Yes | RUES2 | Perc | 89% | 88% | 10.9% | 4.2% | 79.4 | 75.7 | 35.4 | 44.9 | Survival |
| 16 | 8.8 | 34.0 | Yes | RUES2 | Perc | 88% | 85% | 14.6% | 0.7% | 74.2 | 68.6 | 43.9 | 7.6 | Survival |
| 18 | 12.7 | 32.5 | Yes | RUES2 | Perc | 94% | 75% | 9.3% | 3.7% | 69.0 | 78.2 | 1.6 | 1.6 | Survival |
| Avg ± SEM | 8.8 ± 0.6 | 33.7 ± 0.5 | | | | 90 ± 1% | 87 ± 2% | 11.7 ± 1.1% | 2.3 ± 0.7% | 90.3 ± 9.7 | 79 ± 2.9 | 39.5 ± 9.2 | 26.5 ± 13.4 | |

No Treatment

| | | | | | | | | | | | | | | |
|---|---|---|---|---|---|---|---|---|---|---|---|---|---|---|
| 1 | 8.9 | 32.0 | No | H7 | Surg | 98% | 88% | — | — | 328.2 | n/a | 100.0 | n/a | Primary endpoint day 7 |
| | | | | | | | | | | | | | | (VF) |
| 2 | 10.5 | 32.0 | Yes | H7 | Surg | 98% | 90% | — | — | 120.2 | n/a | 62.7 | n/a | Primary endpoint day 18 |
| | | | | | | | | | | | | | | (VF) |
| 11 | 9.4 | 33.5 | Yes | RUES2 | Perc | 82% | 87% | 9.7% | 1.1% | 165.6 | n/a | 94.0 | n/a | Primary endpoint day 12 |
| | | | | | | | | | | | | | | (EA) |
| 12 | 9.5 | 35.0 | Yes | RUES2 | Perc | 86% | 90% | 13.2% | 1.0% | n/a | n/a | n/a | n/a | Primary endpoint day 5 |
| | | | | | | | | | | | | | | (EA) |
| 13 | 9.7 | 35.5 | Yes | RUES2 | Perc | 87% | 83% | 16.5% | 1.7% | 162.0 | 82.6 | 95.9 | 1.9 | Survival |
| 14 | 9.0 | 33.0 | Yes | RUES2 | Perc | 87% | 90% | 4.6% | 9.1% | n/a | n/a | n/a | n/a | Primary endpoint day 6 |
| | | | | | | | | | | | | | | (EA/HF) |
| 17 | 7.0 | 33.0 | Yes | RUES2 | Perc | 98% | 70% | 13.5% | 0.4% | 106.0 | 115.2 | 49.1 | 75.7 | Survival |
| 19 | 6.4 | 33.5 | Yes | RUES2 | Perc | 92% | 74% | 5.4% | 3.6% | 98.5 | 84.3 | 71.5 | 51.7 | Survival |
| Avg ± SEM | 8.8 ± 0.5 | 33.4 ± 0.4 | | | | 91 ± 2% | 84 ± 3% | 10.5 ± 2% | 2.8 ± 1.3% | 163.4 ± 34.9 | 94 ± 10.6 | 78.9 ± 8.5 | 43.1 ± 21.7 | |
| P-value^{†} | 0.97 | 0.73 | | | | 0.77 | 0.31 | 0.59 | 0.74 | 0.03 | 0.09 | 0.01 | 0.52 | |

Sham Transplant

| | | | | | | | | | | | | | | |
|---|---|---|---|---|---|---|---|---|---|---|---|---|---|---|
| 9 | 8.30 | 33.5 | Yes | n/a | Perc | n/a | n/a | — | n/a | 71.5 | 67.6 | 0.8 | 0.0 | Survival |
| 10 | 7.77 | 33.0 | Yes | n/a | Perc | n/a | n/a | — | n/a | 76.5 | 69.4 | 1.2 | 0.8 | Survival |
| Avg ± SEM | 8.0 ± 0.3 | 33.3 ± 0.3 | | | | | | | | 74 ± 2.5 | 68.5 ± 0.9 | 1.0 ± 0.2 | 0.4 ± 0.4 | |

^{†}Treatment vs No treatment
Abbreviations: bpm, beats per minute; EA, engraftment arrhythmia; hESC-CM, human embryonic stem cell-derived cardiomyocytes; HF, heart failure; HR, heart rate; MI, myocardial infarction; surg, surgery; PCP, pneumocystis pneumonia; pCMV, porcine cytomegalovirus; perc, percutaneous; VF, ventricular fibrillation

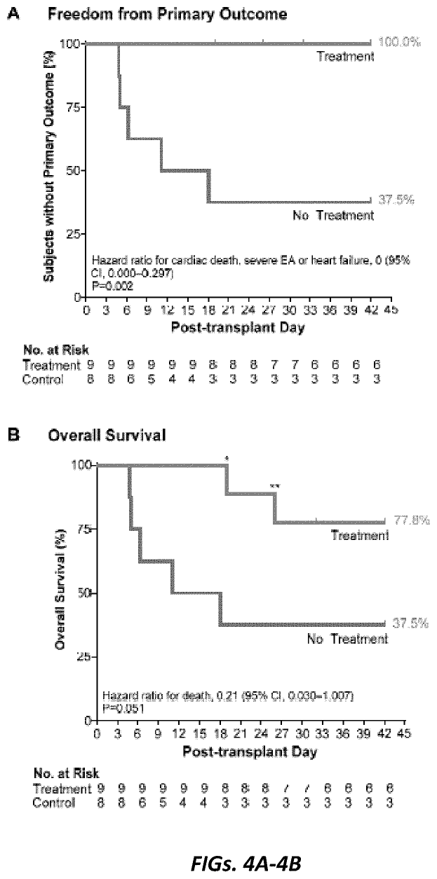

As detailed in the ‘Methods’, treated animals received bolus and maintenance doses of amiodarone, and ivabradine was given as needed to keep heart rates <150 bpm. All treatment subjects (100%) survived without the primary cardiac endpoint compared to 3/8 (37.5%) of untreated subjects (FIG. 4A). The hazard ratio of the primary endpoint was 0.000 (95% CI, 0.000-0.297; p=0.002) with antiarrhythmic treatment. Of note, two of the treatment subjects (3 and 6) experienced non-cardiac deaths at post-transplant days 19 and 26 due to immunosuppression-related complications (Pneumocystis pneumonia and porcine cytomegalovirus, respectively). Intention-to-treat analysis of overall survival also favored the treatment cohort with hazard ratio of 0.212 (95% CI, 0.030-1.007; p=0.051) (FIG. 4B).

Suppression of Tachycardia and Arrhythmia Burden

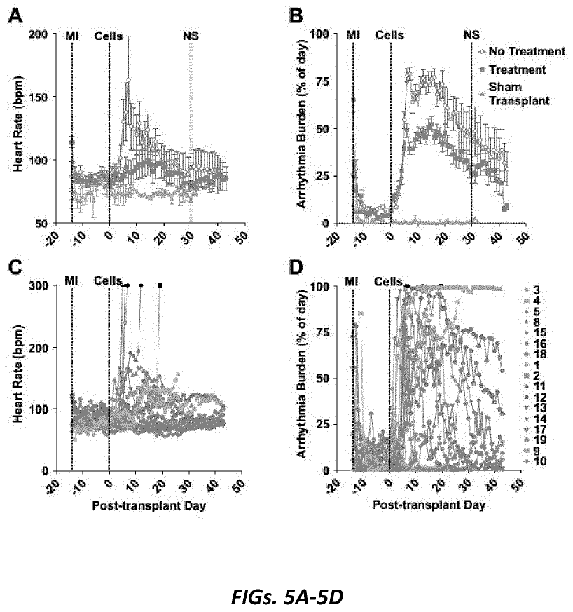

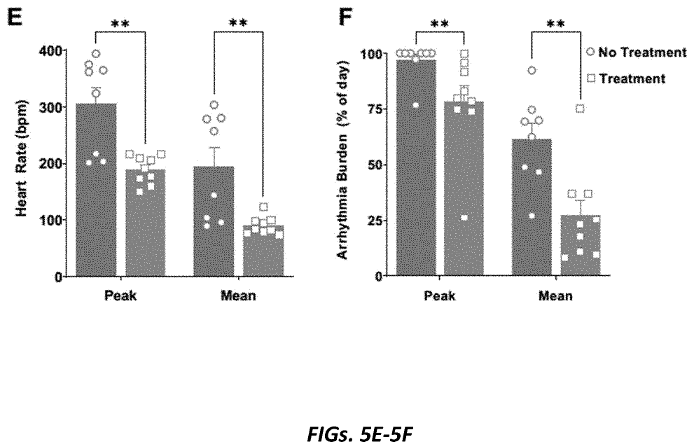

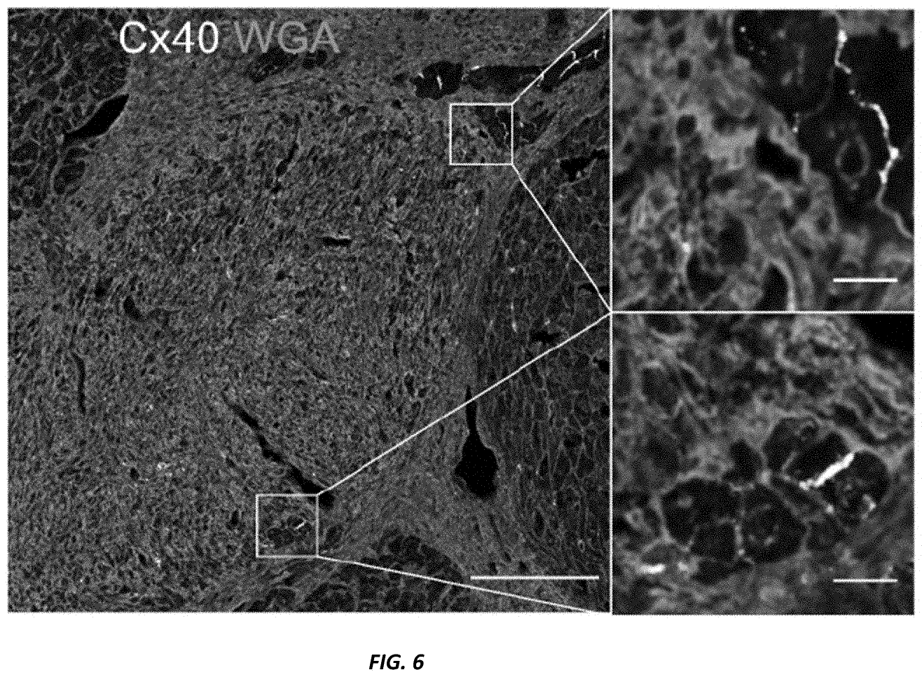

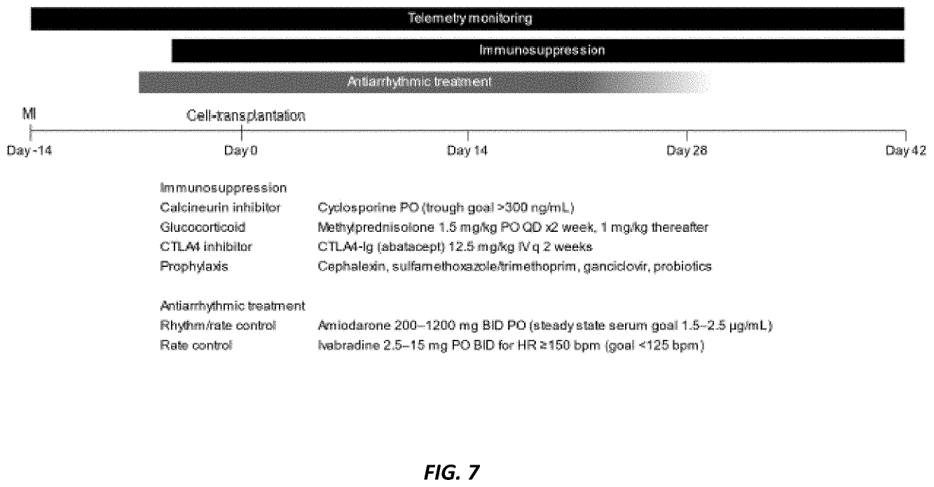

Pooled and individual subject-level data of heart rate and arrythmia burden are provided in FIGS. 5A,5B and FIGS. 5C,5D, respectively. The average heart rate was significantly lower with antiarrhythmic treatment compared to no treatment. Mean heart rates peaked at post-transplantation day 7 in untreated animals at 163±35 bpm, when in the treated group heart rates averaged 90±10 bpm (p=0.03) (Table 2 and FIG. 5). Heart rate in the treated animals was not significantly different than the normal resting heart rate prior to MI and transplant (84±1 bpm, p=0.21). Following transplantation, peak heart rate averaged 305±29 beats/min in untreated animals, whereas treatment significantly restricted peak heart rate to 185±9 beats/min (p=0.001) (FIG. 5E). The inventors defined arrhythmia burden as the percentage of the day spent in arrhythmia. Treatment reduced peak arrhythmia burden from 96.8±2.9% to 76.5±7.9% (p=0.03) (FIG. 5F). No differences in heart rate or arrhythmia burden were noted at post-transplant day 30, as the majority of arrhythmia had resolved irrespective of treatment (FIGS. 5A & 5B) (p=0.09 and p=0.52, respectively).

Antiarrhythmic treatment was safely discontinued by day 30 in all treatment subjects who achieved electrical maturation without recrudescence of arrhythmia (FIG. 5). Two treated and two untreated subjects (3, 4 and 15, 17, respectively) failed to mature electrically and exhibited significant arrhythmia at the end of study. In these four animals, heart rates were well controlled irrespective of treatment, and they survived until the study's completion. Average serum amiodarone was sub-therapeutic at 0.42±0.12 μg/ml within 1 week of discontinuation (FIG. 8).

Graft Interaction with Host Purkinje Conduction System

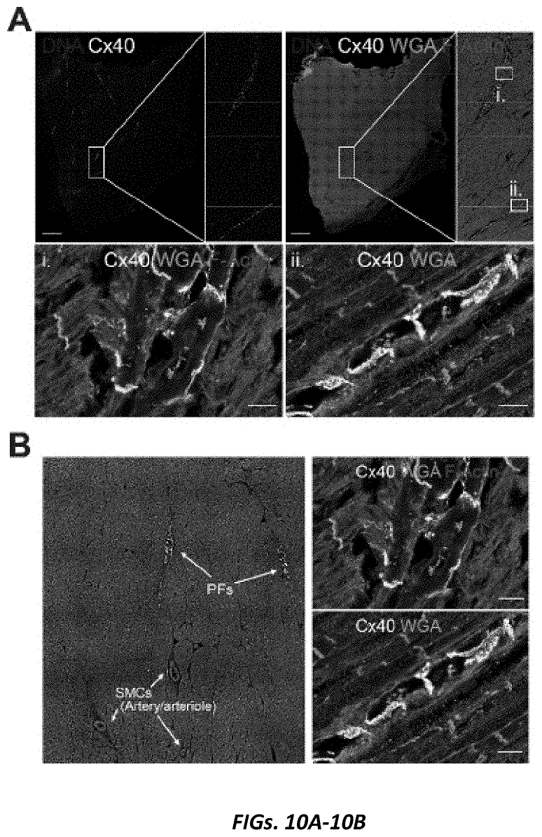

The narrow-complex tachycardia that resembles accelerated junctional rhythm (FIG. 2) was not observed in previous monkey studies (8,9) but was common here in the pig. Pigs are known to have an extensive Purkinje fiber network that extends transmurally throughout the ventricular myocardium, whereas in macaques and humans the Purkinje network is subendocardial (20,21). It was hypothesized that the narrow-complex VT resulted from grafts contacting and entraining intramural Purkinje fibers, with retrograde activation to the rest of the ventricle. Histology confirmed the mesh-like network of intramural Purkinje fibers (PFs) throughout the porcine left ventricle (FIG. 10A).

There were multiple examples of hESC-CM grafts in direct contact with these intramural branches of the Purkinje system. (FIG. 6). Connexin 40 (Cx40) immunostaining was used to specifically stain Purkinje fiber gap junctions (20,22), and their identity was confirmed by their reduced myofibril content and the absence of T tubules (FIG. 10B). This supports the hypothesis that the pig's unique Purkinje network anatomy contributes to narrow-complex engraftment arrhythmia.

Intramyocardial transplantation of hPSC-CM is a promising strategy to remuscularize the infarcted heart and restore function (2). Such a therapy to prevent and treat heart failure would be a seminal advance in addressing a large unmet need. Studies in large animals have demonstrated long-term efficacy but also defined a significant safety signal of transient but potentially fatal arrhythmias. As demonstrated in earlier studies (9-11), EA is a predictable complication of cardiac remuscularization therapy for myocardial infarction (23). In the NHP, EA typically presents as a wide-complex tachycardia with a variable electrical axis (8,9), and this was reproduced in the pig recently by Laflamme laboratory (11).

Here the inventors further describe EA as polymorphic and interpret the changes in electrical axis as ectopy originating from different graft foci. Interestingly, in the pig the inventors also observed a narrow-complex VT that alternate with wide-complex tachycardia, a pattern not seen in the NHP. Histology of native and grafted porcine myocardium support the hypothesis that that the wide-complex beats originate from grafts contacting the working-type myocardium with slow conduction, and that the narrow complex beats originate when grafts contact the intramural Purkinje fibers that are diffusely permeate the porcine heart (20,21).

All 17 subjects transplanted with 500×10⁶ hESC-CMs demonstrated significant burden of arrhythmia that, while typically transient, was associated with high mortality in pigs. The inventors observed higher morbidity and mortality related to EA than the recent study by Laflamme and colleagues (11), perhaps reflecting differences in our animal model including use of Yucatan minipigs, percutaneous cell delivery, or our cell product. The inventors experience with this model indicates two primary mechanisms of cardiac morbidity.

Firstly, rapid EA>350 bpm often degenerates to fatal ventricular fibrillation, and secondly, heart failure commonly ensues in pigs with chronic tachycardia >230 bpm (24). Consequently, the primary endpoint included these parameters to limit excessive mortality in our antiarrhythmic trial.

Combined antiarrhythmic treatment with baseline amiodarone and adjunctive ivabradine safely prevented the combined primary endpoint of cardiac death, unstable EA and heart failure in all treated subjects, indicating that the risk of EA may be mitigated through pharmacology. Treatment was associated with significantly decreased peak tachycardia and arrhythmia. Once subjects experienced sustained improvement in arrhythmia burden, termed electrical maturation, antiarrhythmic therapy was successfully withdrawn in all subjects. Thus, short-term amiodarone and ivabradine treatment promoted electrical stability until the grafts became less arrhythmogenic.

The mechanism of benefit for our antiarrhythmic treatment may be related to suppression of automaticity, reducing both heart rate and arrhythmia burden. The drugs were particularly beneficial during the early phase of EA, which carries the greatest risk of deterioration to VF. Electrophysiological studies performed by the inventors and the Laflamme laboratory in NHP (9) and pig (11), respectively, indicates that the etiology of EA is increased focal automaticity, rather than macro-reentry typically observed with clinical ventricular tachycardia (25). As EA became unstable in the untreated animals, heart rates rapidly accelerate to >350 bpm, and the inventors cannot exclude the possibility that this escalation could have a distinct mechanism, e.g. automaticity leading to reentry. This may explain why treatment successfully suppressed unstable and fatal arrhythmias but was unable to prevent EA altogether.

The efficacy of ivabradine to rate-control EA indicates that its pharmacologic target, the I_{f} current carried by the HCN4 channel, which is highly expressed in immature cardiomyocytes and hPSC-CMs (26), may be an important mediator. Ivabradine, by itself, never abrogated EA, indicating that the I_{f} current is a rate-modulator but not the sole source of the arrhythmia. In contrast, amiodarone reduced the burden of EA chronically and clearly restored sinus rhythm in some acute infusion experiments (FIG. 3). Although classified principally as a K+ channel blocker, amiodarone is well-known also to antagonize Na+ channels, Ca2+ channels, and β-adrenergic receptors (27). Thus, it is difficult to gain insights into the mechanism of EA from amiodarone's efficacy. The disappearance of EA coincides with maturation of the stem cell-derived graft (8,9,28) and the inventors have hypothesized that the window of arrhythmogenicity may reflect a period of in vivo graft maturation prior to reaching a state more similar to host myocardium (26,29-33). Additional strategies such as promoting maturation prior to transplanting, gene editing, and modulating host/cell interaction may provide additional means of arrhythmia control. Further investigation of the mechanism underlying EA could be accelerated by the development of higher throughput platforms to perform genetic, pharmacological and electrophysiological studies before phenotyping in large animal models.

Engraftment arrhythmia is the most significant barrier to clinical translation of cardiac remuscularization therapy. The natural history of EA emerging from the NHP and more recent porcine data indicates that, once EA resolves, there is low risk for further arrhythmia. This study provides a proof-of-concept that clinically relevant antiarrhythmic drug treatment can successfully suppress fatal arrhythmias and control tachycardia to achieve electrical quiescence. This could be an important tool toward reaching an acceptable safety profile for clinical development.

While this study demonstrates that EA is responsive to pharmacologic suppression, there are several limitations. It would be useful to perform a longer follow-up to establish the long-term effectiveness of EA mitigation as well as dosing studies to optimize the treatment regimen. The inventors did not randomize enrollment of subjects or assess whether sex is a biological variable. Although they took pains to administer clinically relevant doses of amiodarone and ivabradine, the inventors cannot exclude the possibility that EA, itself, is dependent on the dose of cells transplanted. Future studies will also ideally include functional endpoints to determine mechanical efficacy with background guideline-directed medical therapy such as inhibitors of the renin—angiotensin—aldosterone and β-adrenergic systems.

In sum, this study utilizes a porcine infarction model of cardiac remuscularization therapy where EA was universally observed and associated with significant mortality. Chronic amiodarone treatment combined with adjunctive ivabradine successfully prevented the combined primary endpoint of cardiac death, unstable EA and heart failure. Overall survival was significantly improved with antiarrhythmic treatment and associated with heart rate and rhythm control. The mechanisms of engraftment arrhythmia remain poorly understood and merit concerted scientific inquiry.

Clinical Perspectives

Heart failure remains a significant cause of morbidity and mortality following myocardial infarction (MI). Cardiac remuscularization with transplantation of pluripotent stem cell-derived cardiomyocytes is a promising preclinical therapy to restore function. Recent large animal data, however, have revealed a significant risk of engraftment arrhythmia (EA). The present study provides proof-of-concept evidence that a combination of amiodarone and ivabradine can effectively prevent EA-related mortality and suppresses tachycardia and arrhythmia burden. Thus, pharmacologic suppression of EA may be a viable strategy to improve safety and allow further clinical development of cardiac remuscularization therapy.

### REFERENCES

1. Collaborators GBDCoD. Global, regional, and national age-sex-specific mortality for 282 causes of death in 195 countries and territories, 1980-2017: a systematic analysis for the Global Burden of Disease Study 2017. Lancet 2018; 392:1736-1788.
2. Nakamura K, Murry C E. Function Follows Form—A Review of Cardiac Cell Therapy. Circ J 2019; 83:2399-2412.
3. Caspi O, Huber I, Kehat I et al. Transplantation of human embryonic stem cell-derived cardiomyocytes improves myocardial performance in infarcted rat hearts. J Am Coll Cardiol 2007; 50:1884-93.
4. Laflamme M A, Chen K Y, Naumova A V et al. Cardiomyocytes derived from human embryonic stem cells in pro-survival factors enhance function of infarcted rat hearts. Nat Biotechnol 2007; 25:1015-24.
5. Shiba Y, Fernandes S, Zhu W Z et al. Human ES-cell-derived cardiomyocytes electrically couple and suppress arrhythmias in injured hearts. Nature 2012; 489:322-5.
6. van Laake L W, Passier R, Monshouwer-Kloots J et al. Human embryonic stem cell-derived cardiomyocytes survive and mature in the mouse heart and transiently improve function after myocardial infarction. Stem Cell Res 2007; 1:9-24.
7. Shiba Y, Filice D, Fernandes S et al. Electrical Integration of Human Embryonic Stem Cell-Derived Cardiomyocytes in a Guinea Pig Chronic Infarct Model. J Cardiovasc Pharmacol Ther 2014; 19:368-381.
8. Chong J J, Yang X, Don C W et al. Human embryonic-stem-cell-derived cardiomyocytes regenerate non-human primate hearts. Nature 2014; 510:273-7.
9. Liu Y W, Chen B, Yang X et al. Human embryonic stem cell-derived cardiomyocytes restore function in infarcted hearts of non-human primates. Nat Biotechnol 2018; 36:597-605.
10. Shiba Y, Gomibuchi T, Seto T et al. Allogeneic transplantation of iPS cell-derived cardiomyocytes regenerates primate hearts. Nature 2016; 538:388-391.
11. Romagnuolo R, Masoudpour H, Porta-Sanchez A et al. Human Embryonic Stem Cell-Derived Cardiomyocytes Regenerate the Infarcted Pig Heart but Induce Ventricular Tachyarrhythmias. Stem Cell Reports 2019; 12:967-981.
12. Eschenhagen T, Bolli R, Braun T et al. Cardiomyocyte Regeneration: A Consensus Statement. Circulation 2017; 136:680-686.
13. Lelovas P P, Kostomitsopoulos N G, Xanthos T T. A comparative anatomic and physiologic overview of the porcine heart. J Am Assoc Lab Anim Sci 2014; 53:432-8.
14. van der Spoel T I, Jansen of Lorkeers S J, Agostoni P et al. Human relevance of preclinical studies in stem cell therapy: systematic review and meta-analysis of large animal models of ischaemic heart disease. Cardiovasc Res 2011; 91:649-58.
15. Chen V C, Ye J, Shukla P et al. Development of a scalable suspension culture for cardiac differentiation from human pluripotent stem cells. Stem Cell Res 2015; 15:365-75.
16. Staubli M, Bircher J, Galeazzi R L, Remund H, Studer H. Serum concentrations of amiodarone during long term therapy. Relation to dose, efficacy and toxicity. Eur J Clin Pharmacol 1983; 24:485-94.
17. Mostow N D, Rakita L, Vrobel T R, Noon D L, Blumer J. Amiodarone: correlation of serum concentration with suppression of complex ventricular ectopic activity. Am J Cardiol 1984; 54:569-74.
18. Schindelin J, Rueden C T, Hiner M C, Eliceiri K W. The ImageJ ecosystem: An open platform for biomedical image analysis. Mol Reprod Dev 2015; 82:518-29.
19. Deroulers C, Ameisen D, Badoual M, Gerin C, Granier A, Lartaud M. Analyzing huge pathology images with open source software. Diagn Pathol 2013; 8:92.
20. Garcia-Bustos V, Sebastian R, Izquierdo M, Molina P, Chorro F J, Ruiz-Sauri A. A quantitative structural and morphometric analysis of the Purkinje network and the Purkinje-myocardial junctions in pig hearts. J Anat 2017; 230:664-678.
21. Panescu D, Kroll M, Brave M. Limitations of animal electrical cardiac safety models. Annu Int Conf IEEE Eng Med Biol Soc 2014; 2014:6483-6.
22. Pallante B A, Giovannone S, Fang-Yu L et al. Contactin-2 expression in the cardiac Purkinje fiber network. Circ Arrhythm Electrophysiol 2010; 3:186-94.
23. Yu J K, Franceschi W, Huang Q, Pashakhanloo F, Boyle P M, Trayanova N A. A comprehensive, multiscale framework for evaluation of arrhythmias arising from cell therapy in the whole post-myocardial infarcted heart. Sci Rep 2019; 9:9238.
24. Chow E, Woodard J C, Farrar D J. Rapid ventricular pacing in pigs: an experimental model of congestive heart failure. Am J Physiol 1990; 258:H1603-5.
25. Josephson M, Marchlinski F, Buxton A et al. Electrophysiologic basis for sustained ventricular tachycardia: role of reentry. Tachycardias: Mechanisms, Diagnosis, Treatment Philadelphia, Pa.: Lea and Febiger 1984:305-323.
26. Karbassi E, Fenix A, Marchiano S et al. Cardiomyocyte maturation: advances in knowledge and implications for regenerative medicine. Nat Rev Cardiol 2020.
27. Waks J W, Zimetbaum P. Antiarrhythmic Drug Therapy for Rhythm Control in Atrial Fibrillation. J Cardiovasc Pharmacol Ther 2017; 22:3-19.
28. Kadota S, Pabon L, Reinecke H, Murry C E. In Vivo Maturation of Human Induced Pluripotent Stem Cell-Derived Cardiomyocytes in Neonatal and Adult Rat Hearts. Stem Cell Reports 2017; 8:278-289.
29. Marchiano S, Bertero A, Murry C E. Learn from Your Elders: Developmental Biology Lessons to Guide Maturation of Stem Cell-Derived Cardiomyocytes. Pediatr Cardiol 2019; 40:1367-1387.
30. Guo Y, Pu W T. Cardiomyocyte Maturation: New Phase in Development. Circ Res 2020; 126:1086-1106.
31. Kannan S, Kwon C. Regulation of cardiomyocyte maturation during critical perinatal window. J Physiol 2020; 598:2941-2956.
32. Maroli G, Braun T. The long and winding road of cardiomyocyte maturation. Cardiovasc Res 2020.
33. Ichimura H, Kadota S, Kashihara T et al. Increased predominance of the matured ventricular subtype in embryonic stem cell-derived cardiomyocytes in vivo. Sci Rep 2020; 10:11883.
34. El-Nachef D, Oyama K, Wu Y Y, Freeman M, Zhang Y, MacLellan WR. Repressive histone methylation regulates cardiac myocyte cell cycle exit. J Mol Cell Cardiol 2018; 121:1-12.

### Example 2: Pharmacological Management of Engraftment Arrhythmias

Approach: Pre-treatment of RUES2-CMs (ESC derived cardiomyocytes) with amiodarone before cell injection. Ivabradine, and optionally amiodarone can be administered systemically after cell administration as needed to further manage engraftment arrhythmias.

In this Example, the pig model of cardiac injury and in vitro-differentiated cardiomyocyte administration as described in Example 1 can be used. Cryogenically preserved, in vitro-differentiated cardiomyocytes, including, for example, porcine or human cardiomyocytes, are thawed and incubated as a single cell suspension, e.g., in defined medium, with amiodarone at, e.g., 5 to 10 μg/ml, which mimics therapeutic serum trough levels of ˜1.5-4.0 ug/mL. Differing concentrations or dosages of amiodarone, e.g., in the range of 0.3 to 10 μg/ml (such as, but not limited to 0.3 μg/ml, 1.0 μg/ml, 2.0 μg/ml, 3.0 μg/ml, 4.0 μg/ml, 5.0 μg/ml, 6.0 μg/ml, 7.0 μg/ml, 8.0 μg/ml, 9.0 μg/ml or 10.0 μg/ml) are also contemplated. Incubation for different times, e.g., 2 hr, 1 hr, or 30 minutes can be used to determine optimal efficacy associated, for example, with lowest toxicity. Alternative incubation times can range, for example, from 36, 30, 24, 18, 12, 6, 3, 2 or 1 hour, 30 minutes, 15 minutes, or 5 minutes before administration of, for example, 5×10⁸ or more cells per animal to cardiac tissue of the pig.

Pre-incubation can be performed, for example, under varying conditions, e.g., 0° C., 4° C., room temperature or 37° C. Levels of amiodarone pre-loaded into the cells in this manner can be measured, e.g., using mass spectroscopy. Cell viability can be monitored using standard techniques, and dosages and times adjusted appropriately.

In an alternative embodiment, cells treated at a concentration, for a time and under conditions as described herein can be admixed or suspended with a cryopreservative as known in the art or as described herein (e.g., CRYOSTOR-10′ cryopreservative) in an amount sufficient to preserve viability upon freezing, followed by freezing for cryogenic storage. The cells can then be thawed prior to administration to an animal or subject as described, generally, but not necessarily following removal of the cryopreservative.

In some embodiments, the pre-treated cells can be admixed with or suspended in a matrix or associated with a scaffold as described herein, and the scaffold or matrix can also include amiodarone, for example to further provide a depot for sustained, prolonged or extended delivery of amiodarone post-transplant.

Ivabradine can be administered post transplant as necessary to manage tachycardia or arrhythmia as described in Example 1 or elsewhere herein. Additional amiodarone can be administered, for example, orally or intravenously, following transplant of the cells. Dosages of adjunctive ivabradine and amiodarone for use when administered cardiomyocytes have been pre-treated with amiodarone as described herein can be, for example, as set out in Table 1. Alternatively, it is contemplated that pre-treatment of cardiomyocytes with amiodarone can reduce the dosages or duration of adjunctive anti-arrhythmia drugs necessary to control engraftment arrhythmia in cardiomyocyte transplant recipients.

Cardiac activity and rhythm will be monitored, for example, as described in Example 1. It is expected that pre-treatment of in vitro-differentiated cardiomyocytes with amiodarone will reduce engraftment arrhythmia normally associated with or caused by administration or transplant of such cells, e.g., by at least 10%. It is expected that such pre-treatment will reduce engraftment arrhythmia burden by one or more of delayed onset, fewer hours of arrhythmia per day, shorter duration of arrhythmia, and reduced peak heart rate relative to engraftment arrhythmia burden associated with or caused by transplant of untreated cardiomyocytes.

It is noted that preliminary studies in mice can also be performed in an analogous manner, and may permit, for example, the initial determination of preferred conditions for pre-treatment. Mice do not generally experience engraftment arrhythmia, but impacts of pre-treatment on engraftment efficiency or graft function can be evaluated in the mouse model; i.e., one can evaluate whether pre-treated cells have similar engraftment compared to control, non-treated cells. Larger treatment groups are feasible with the mouse model, e.g., 12 animals per treatment group. It can also be evaluated in the mouse system whether washing of amiodarone-loaded cells prior to injection has an impact on resulting engraftment or function. Engraftment of human cardiomyocytes in this model can be monitored, for example, by Alu-PCR.

1. A transplant composition comprising in vitro-differentiated cardiomyocytes and amiodarone.

2. The composition of claim 1, further comprising a cryopreservative in an amount sufficient to protect viability of the cells upon freezing.

3. The composition of claim 1, wherein the cardiomyocytes are differentiated from embryonic stem cells, induced pluripotent stem (iPS) cells, or obtained by direct reprogramming of non-cardiomyocytes or the cell cycle-activation of pre-existing cardiomyocytes.

4. The composition of claim 3, wherein the iPS cells are derived from a subject who will receive the transplant composition.

5. The composition of claim 1, wherein the amiod3arone is present at a concentration of 0.3 to 10 μg/ml of culture medium, inclusive.

6. The composition of claim 2, wherein the cryopreservative is selected from dimethyl sulfoxide (DMSO), glycerol, sucrose, dextrose, trehalose and polyvinylpyrrolidone.

7. The composition of claim 1, further comprising a scaffold of either synthetic or natural material or an extracellular matrix composition.

8. The composition of claim 7, wherein the scaffold or extracellular matrix composition comprises one or more of a synthetic hydrogel, hyaluronic acid, proteoglycan, collagen, fibronectin, vitronectin, and fibrin.

9. A method of preparing a transplant composition, the method comprising:
a) contacting in vitro-differentiated cardiomyocytes with amiodarone;
b) contacting the amiodarone-contacted cardiomyocytes of (a) with a cryopreservative in a concentration sufficient to protect viability of the cells upon freezing; and
c) freezing the cardiomyocytes resulting from step (b), whereby a transplant composition is prepared.

10. The method of claim 9, wherein the cardiomyocytes are differentiated from induced pluripotent stem (iPS) cells, embryonic stem cells, by direct reprogramming of non-cardiomyocytes, or by cell cycle induction of cardiomyocytes.

11. The method of claim 9, wherein the cardiomyocytes are contacted with amiodarone at a concentration of 0.3 to 10 μg/ml of culture medium, inclusive.

12. The method of claim 9, wherein step (a) comprises contacting the in vitro-differentiated cardiomyocytes with amiodarone for 0-24 hours before step (b).

13. A method of transplanting cardiomyocytes for engraftment in a subject in need thereof, the method comprising:
a) receiving in vitro-differentiated cardiomyocytes, wherein the cardiomyocytes have been contacted with amiodarone; and
b) administering the cardiomyocytes to cardiac tissue of the subject.

14. The method of claim 13, wherein the in vitro-differentiated cardiomyocytes are differentiated from embryonic stem cells or iPS cells, obtained by direct reprogramming of non-cardiomyocytes or the cell cycle-activation of pre-existing cardiomyocytes.

15. The method of claim 13, further comprising administering ivabradine to the subject.

16. The method of claim 13, further comprising administering amiodarone to the subject.

17. The method of claim 13, wherein the cardiomyocytes have been contacted with amiodarone for 0-24 hours before step (a).

18. The method of claim 13, wherein engraftment arrhythmia burden following administering step (b) is reduced relative to that occurring when a preparation of in vitro-differentiated cardiomyocytes that have not been contacted with amiodarone is administered to a subject.

19. The method of claim 13, wherein the cardiomyocytes are administered in admixture with a scaffold or extracellular matrix composition.

20. The method of claim 19, wherein the scaffold or extracellular matrix composition comprises one or more of a synthetic hydrogel, hyaluronic acid, proteoglycan, collagen, fibronectin, vitronectin, and fibrin.
